(19) United States
(12) Patent Application Publication
(10) Pub. No.: US 2026/0116962 A1
(43) Pub. Date: Apr. 30, 2026

(54) PLK1 INHIBITOR IN COMBINATION WITH ANTI-ANGIOGENICS FOR TREATING METASTATIC CANCER

(71) Applicant: CARDIFF ONCOLOGY, INC., San Diego, CA (US)

(72) Inventors: Maya Ridinger, San Diego, CA (US); Tod Smeal, San Diego, CA (US); Mark Erlander, San Diego, CA (US)

(21) Appl. No.: 19/373,225

(22) Filed: Oct. 29, 2025

Related U.S. Application Data
(60) Provisional application No. 63/714,066, filed on Oct. 30, 2024.
(60) Provisional application No. 63/741,728, filed on Jan. 3, 2025.
(60) Provisional application No. 63/778,966, filed on Mar. 27, 2025.

(51) Int. Cl. C07K 16/22; A61K 31/4745; A61K 31/513; A61K 31/519; A61K 31/555; A61K 39/00; A61P 35/04

(52) U.S. Cl. CPC C07K 16/22; A61K 31/4745; A61K 31/513; A61K 31/519; A61K 31/555; A61P 35/04; A61K 2039/505; A61K 2039/545

(57) ABSTRACT

Provided include methods, compositions and kits for treating metastatic cancer in a subject. The method can comprise administering to a metastatic colorectal cancer patient an effective amount of a PLK1 inhibitor (for example, onvansertib) and an effective amount of bevacizumab, wherein the patient has not received any treatment comprising bevacizumab within at least three months prior to the administration of the PLK1 inhibitor and bevacizumab in a manner sufficient to reduce or inhibit progression of the metastatic cancer.

### RELATED APPLICATIONS

This application claims the benefit under 35 U.S.C. § 119(e) of U.S. Provisional Patent Application Ser. No. 63/714,066, filed Oct. 30, 2024, U.S. Provisional Patent Application Ser. No. 63/741,728, filed Jan. 3, 2025, and U.S. Provisional Patent Application Ser. No. 63/778,966, filed Mar. 27, 2025. The content of each of these related applications is incorporated herein by reference in its entirety for all purposes.

### BACKGROUND

### Field

The present disclosure relates generally to the field of treatment for cancer.

### Description of the Related Art

The Polo-like kinase 1 (PLK1) is a serine/threonine kinase and the most well characterized member of this family of 5 closely related regulatory proteins. PLK-1 is a master regulator of mitosis via its control of the entry and progression of cells into and through mitosis. PLK1 performs several important functions throughout mitotic (M) phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis. PLK1 plays a key role in centrosome functions and the assembly of bipolar spindles. PLK1 controls kinetochore interactions with the spindle microtubules that is required for successful separation and segregation chromatids to the appropriate mother and daughter cells. PLK1 also acts as a negative regulator of p53 family members leading to ubiquitination and subsequent degradation of p53/TP53, inhibition of the p73/TP73 mediated pro-apoptotic functions and phosphorylation/degradation of bora, a cofactor of Aurora kinase A. During the various stages of mitosis PLK1 localizes to the centrosomes, kinetochores and central spindle. PLK1 is aberrantly overexpressed in a variety of human cancers and is correlated with cellular proliferation and poor prognosis.

The most advanced stage of cancer is stage IV, which is defined by the cancer spreading to distant parts of the body from where the cancer first originated (e.g., metastasis). Angiogenesis is necessary for metastasis to occur and several anti-angiogenics have been approved for use in humans to treat metastatic cancers. However, according to the Centers for Disease Control and Prevention, about one in every five deaths in the United States is still due to cancer. There is still a need for more effective treatments for advanced (e.g., metastatic) cancers.

### SUMMARY

Disclosed herein include methods of treating cancer, including a metastatic cancer. Provided herein includes a method of treating a metastatic colorectal cancer, comprising: administering to a patient with a metastatic colorectal cancer (mCRC) an effective amount of onvansertib and an effective amount of bevacizumab, wherein the patient has not received any treatment comprising bevacizumab within at least three months prior to the administration of onvansertib and bevacizumab. In some embodiments, the patient has not received any treatment comprising bevacizumab within about three months or about four months prior to the administration of onvansertib and bevacizumab. In some embodiments, the patient has not received any treatment comprising bevacizumab within about three months or about twelve months prior to the administration of onvansertib and bevacizumab. In some embodiments, the patient has received at least one treatment comprising bevacizumab more than three months prior to the administration of onvansertib and bevacizumab. In some embodiments, the patient has received at least one treatment (e.g., a cancer treatment) not comprising bevacizumab. In some embodiments, the at least one treatment not comprising bevacizumab occurs within about three months prior to the administration of onvansertib and bevacizumab.

The method can, for example, further comprise at least three months after the administration of onvansertib and bevacizumab has ended, administrating at least one additional treatment comprising bevacizumab to the patient. The at least one additional treatment can comprise bevacizumab and onvansertib. In some embodiments, any two additional treatments comprising bevacizumab are separated by a time period of at least three months. In some embodiments, the patient has not received any treatment comprising bevacizumab and a chemotherapy containing fluorouracil within at least three months prior to the administration of onvansertib and bevacizumab. The metastatic colorectal cancer can be, for example, a mutated metastatic colorectal cancer having one or more mutations in the RAS gene. In some embodiments, the metastatic colorectal cancer is KRAS-mutated metastatic colorectal cancer, NRAS-mutated metastatic colorectal cancer, HRAS-mutated metastatic colorectal cancer, or a combination thereof. In some embodiments, the administration of onvansertib and bevacizumab is performed in combination with a chemotherapy containing fluorouracil. The chemotherapy containing fluorouracil can be, e.g., FOLFOX, FOLFIRI, 5-fluorouuracil (5-FU), FOLFIRINOX, FOLFOXIRI, or a combination thereof.

In some embodiments, the administration comprises at least two treatment cycles of the administration of onvansertib and bevacizumab, and optionally wherein two of the treatment cycles are in consecutive. In some embodiments, onvansertib is administered on at least four days in a treatment cycle and bevacizumab is administered once every week or two weeks during a treatment cycle. The treatment cycle can be, e.g., about 21 to 28 days.

Onvansertib and bevacizumab can be administered to the patient simultaneously, separately, or sequentially. In some embodiments, the administration of onvansertib is oral administration, and the administration of bevacizumab is intravenous administration. In some embodiments, onvansertib is administered at 12 mg/m²-90 mg/m², optionally at 15 mg/m²-30 mg/m². In some embodiments, onvansertib is administered at 5-50 mg (e.g., at 10-30 mg), including at 10 mg, 15 mg, 20 mg, 25 mg, or 30 mg. In some embodiments, bevacizumab is administered at 1 mg/kg-20 mg/kg. In some embodiments, onvansertib and bevacizumab are administered to the patient in combination with FOLFIRI. In some embodiments, onvansertib and bevacizumab are administered to the patient in combination with FOLFOX. Onvansertib, bevacizumab, and FOLFIRI or FOLFOX can be administered to the patient simultaneously, separately or sequentially.

The method can further comprise administering to the patient one or more additional cancer therapeutics or therapies. Non-limiting examples of the one or more additional cancer therapeutics or therapies include FOLFIRI, abiraterone, FOLFOX, an anti-EGFR agent, a KRAS-directed inhibitor, gemcitabine, abraxane, nanoliposomal irinotecan, 5-FU, or a combination thereof. The method can further comprises administering a chemotherapy to the patient. In some embodiments, the chemotherapy comprises a treatment using FOLFIRI, FOLFOX, gemcitabine, abraxane, nanoliposomal irinotecan, 5-fluorouuracil, FOLFIRINOX, FOLFOXIRI, or a combination thereof.

The method can further comprise determining cancer status of the patient. In some embodiments, onvansertib and bevacizumab synergistically improves objective response rate (ORR) and/or progression free survival (PFS) in the patient. In some embodiments, the patient achieves about 2-fold, 3-fold, 4-fold or 5-fold higher ORR, compared to patients who have received the treatment comprising bevacizumab within about three months prior to the administration, optionally, within about 2 months or one month prior to the administration.

The method can further comprise identifying the patient as having the metastatic colorectal cancer and as not having received any treatment comprising bevacizumab within at least three months prior to the administration of onvansertib and bevacizumab. The method can further comprise excluding patients who have received a prior treatment comprising bevacizumab within at least three months from receiving the onvansertib and bevacizumab treatment. The method can further comprise identifying patients who have received a prior treatment comprising bevacizumab within at least three months. In some embodiments, the patient is human.

### BRIEF DESCRIPTION OF THE DRAWINGS

FIG. 1 depicts a graphical summary of the proposed mechanisms of onvansertib (Onv) and bevacizumab (Bev) combination therapy in Bev-naive tumors. The combination of Onv and Bev effectively inhibits tumor cell survival, proliferation, and angiogenesis through the indicated mechanisms.

FIG. 2 depicts antitumor activity according to prior bevacizumab treatment. Panel A shows best percentage change from baseline in target lesions. The dashed line at −30% change represents the RECIST v1.1 cutoff to define PR. Confirmed responses are indicated. Panel B shows Kaplan-Meier curve of the PFS. Bev, bevacizumab, CR, complete response; PFS, progression-free survival; PR, partial response.

FIG. 3 shows association between time since first-line bevacizumab treatment (Bev 1L) and clinical benefit in bevacizumab-exposed. Panel A shows time since treatment with bevacizumab in first-line (Bev 1L) according to best response. Data analyzed by one-way ANOVA with Tukey multiple comparison test. ** and *** correspond to p-value <0.01 and <0.001 respectively; ns:non-significant. Panel B shows progression-free survival (PFS) of patients with time since Bev 1L>3.2 months and time since Bev 1L≤3.2 months; p-value (p) is indicated.

FIG. 4 shows association between time since first-line bevacizumab treatment (Bev 1L) and clinical benefit in bevacizumab-exposed. Panel A shows time since treatment with bevacizumab in first-line (Bev 1L) according to best response. Data analyzed by one-way ANOVA with Tukey multiple comparison test. ** and *** correspond to p-value <0.01 and <0.001 respectively; ns:non-significant. Panel B shows progression-free survival (PFS) of patients with time since Bev 1L>4 months and time since Bev 1L≤4 months; p-value (p) is indicated.

FIG. 5 depicts a flow diagram of the phase Ib and phase II. ^{“a”} indicates drugs were given once daily on days 1-5 and 15-19 of a 28-day cycle. ctDNA, circulating tumor DNA; Onv, onvansertib.

FIG. 6A-FIG. 6B show transcriptomic changes associated with bevacizumab treatment. FIG. 6A shows a schematic representation of analysis workflow. FIG. 6B shows gene set enrichment analysis of differentially expressed genes in patients treated with oxaliplatin+bevacizumab (bevacizumab-exposed) compared with oxaliplatin (bevacizumab-naïve). Bar plots show normalized enrichment scores (NES) of pathways significantly upregulated in bevacizumab-exposed patients (FDR p-value<0.05). Pathways in bold indicate pathways potentially involved in onvansertib and bevacizumab resistance. Bev, bevacizumab; FDR, false discovery rate; mCRC, metastatic colorectal cancer; NES, normalized enrichment scores.

FIG. 7A-FIG. 7E show antitumor activity of onvansertib and bevacizumab in KRAS-mutant CRC xenografts and regulation of the hypoxia pathway by PLK1. FIG. 7A-FIG. 7C show tumor-bearing mice were treated with vehicle, onvansertib and/or bevacizumab for 30-33 days. FIG. 7A shows tumor growth curves. FIG. 7B shows SW620 and LoVo tumor photographs, scale bar: 10 mm. FIG. 7C shows representative immunohistochemical images of SW620 tumors stained with anti-CD31 antibody (LoVo tumors are shown in FIG. 10B), scale bar: 100 μm. CD31+blood vessels were quantified; graphs represent the mean score±SEM for each treatment group (n=6/group). Statistical analyses were performed using one-way ANOVA. **p<0.01, ***p<0.001, ****p<0.0001. For FIG. 7D-FIG. 7E, KRAS-mutant CRC cell lines were treated with DMSO or onvansertib at the indicated doses for 20 hours and then exposed to hypoxia for 4 hours. FIG. 7D shows representative immunoblot images of HIF1α and j-actin and quantification of HIF1α expression normalized to j-actin and normoxia control sample. Graphs represent mean±SEM of ≥3 independent experiments. FIG. 7E shows dot plot showing hallmarks of cancer hypoxia and glycolysis gene set enrichment analyses from comparisons of the indicated groups. The size of each dot corresponds to significance (−log₁₀(P)). ANOVA, analysis of variance; Bev, bevacizumab; FDR, false discovery rate; HIF1α, hypoxia-inducible factor 1α; Hx, hypoxia; Nx, normoxia; Onv, onvansertib; PLK1, polo-like kinase 1; P, p-valuee.

FIG. 8A-FIG. 8B show antitumor activity (n=53). FIG. 8A shows best percentage change from baseline in target lesions. The dashed line at −30% change represents the RECIST v1.1 cutoff to define partial response. Confirmed responses are indicated. FIG. 8B shows Kaplan-Meier curve of the PFS. CR, complete response; PR, partial response; PFS, progression-free survival.

FIG. 9A-FIG. 9B show association between changes in KRAS-mutant ctDNA and clinical benefit. FIG. 9A shows percentage change in KRAS-mutant ctDNA from baseline after 1 cycle of treatment according to best response. Data analyzed by unpaired t-test with Welch correction, **** corresponds to p-value<0.0001. FIG. 9B shows PFS of patients who exhibited a ≥90% decrease in KRAS-mutant ctDNA after 1 cycle of treatment and in patients with less than 90% decrease. CR, complete response; ctDNA, circulating tumor DNA; HR, hazard ratio; PD, progressive disease; PFS, progression-free survival; PR, partial response; SD, stable disease.

FIG. 10A-FIG. 10D show antitumor activity of onvansertib and bevacizumab in KRAS-mutant CRC xenografts, related to FIG. 7A-FIG. 7E. SW620 and LoVo tumors after treatment with either vehicle, onvansertib (Onv), and/or bevacizumab (Bev) for 30-33 days. FIG. 10A shows SW620 and LoVo tumor photographs, scale 10 mm. FIG. 10B shows representative immunohistochemical images of LoVo tumors stained with anti-CD31 antibody, scale bar 100 μm. FIG. 10C shows representative immunohistochemical images of SW620 and LoVo tumors stained with anti-BrdU antibody, scale bar 100 μm. Graph represents the % of BrdU+ proliferating tumor cells (mean±SEM) for each treatment group (n=6/group). FIG. 10D shows representative H&E images of SW620 and LoVo tumors, scale bar 50 μm. Tumor cells with apoptotic morphology were scored; graph represents the score (mean±SEM) for each treatment group (SW620: n=9/group, LoVo: n=6/group). Statistical analyses were performed using one-way ANOVA. *p<0.05, **p<0.01, ***p<0.001, ****p<0.0001. ANOVA, analysis of variance; Bev, bevacizumab; H&E, hematoxylin and eosin; Onv, onvansertib.

FIG. 11A-FIG. 11D show regulation of the hypoxia pathway by PLK1, related to FIG. 7A-FIG. 7E. In FIG. 11A-FIG. 11B, KRAS-mutant CRC cells were treated with DMSO or onvansertib at the indicated doses for 20 hours and then exposed to hypoxia or kept in normoxia for 4 hours. FIG. 11A shows a heatmap of hypoxiarelated genes significantly regulated by onvansertib in HCT116 and SW620 cells based on the RNA-seq analysis. FIG. 11B shows expression of hypoxia-related genes in LoVo and DLD-1 cells, assessed by RT-qPCR and normalized to the housekeeping gene RPLP0. Bar graphs represent expression relative to Normoxia_DMSO sample. In FIG. 11C-FIG. 11D, SW620 and HCT116 cells were transfected with non-targeting control siRNA (siNTC) or PLK1 targeting siRNA (siPLK1) for 20 hours and then exposed to hypoxia for 4 hours. Left panel of FIG. 11C shows immunoblot of PLK1, HIF1α and β-actin. Right panel of FIG. 11C shows HIF1α and PLK1 protein expression normalized to β-actin. FIG. 11D shows expression of hypoxia-related genes assessed by RT-qPCR and normalized to the housekeeping gene RPLP0. Bar graphs represent expression relative to Normoxia_siNTC. In FIG. 11B-FIG. 11D, data are shown as mean±SEM of at least 3 independent biological replicates. HIF1α, hypoxia-inducible factor 1α; Hx, hypoxia; Nx, normoxia; Onv, onvansertib; PLK1, polo-like kinase 1; RT-qPCR, real-time quantitative polymerase chain reaction

FIG. 12 shows graphical summary of the proposed mechanisms of onvansertib and bevacizumab combination therapy in bevacizumab-naïve and bevacizumab-exposed tumors. In bevacizumab-exposed tumors, bevacizumab exposure leads to upregulation of mitotic and hypoxia pathways resulting in resistance to both onvansertib and bevacizumab. In contrast, for Bev-naïve tumors (FIG. 1), the combination of Onv and Bev effectively inhibits tumor cell survival, proliferation, and angiogenesis through the indicated mechanisms. Onv, onvansertib; HIF1α, hypoxia-inducible factor 1α; Bev, bevacizumab.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 3 would include 2.7 and 3.3, about 10 would include 9 to 11, about 1000 would include 900 to 1100.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor or a Doctor of Veterinary Medicine, to attempt to cure, or at least ameliorate the effects of, a particular disease or disorder or to prevent the disease or disorder from occurring in the first place. The patient can be an animal. In some embodiments, the patient is a mammal. In some embodiments, the patient is a human.

As used herein, “administration” or “administering” refers to a method of giving a dosage of a pharmaceutically active ingredient to a vertebrate.

As used herein, a “dosage” refers to the combined amount of the active ingredients (e.g., PLK1 inhibitor (e.g., onvansertib) or anti-angiogenics (e.g., bevacizumab)).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, each of the terms “partial response,” “partial remission,” or “PR” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone Marrow transplantation, and immunotherapy. The size of a tumor can be detected by clinical or by radiological means. Tumor-indicating markers can be detected by means well known to those of skill, e.g., ELISA or other antibody-based tests.

As used herein, each of the terms “complete response,” “complete remission,” “complete recovery,” or “CR” refers to a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. However, a “complete response” does not necessarily indicate that the cancer has been cured. A complete response may be followed by a relapse. A complete response of a target lesion includes disappearance of all target lesions and any pathological lymph nodes (whether target or non-target) having reduction in short axis to <10 mm. A complete response of a non-target lesion includes disappearance of all non-target lesions and normalization of tumor marker level (all lymph nodes must be non-pathological in size (<10 mm short axis)). If tumor markers are initially above the upper normal limit, they need to normalize for a patient to be considered in complete clinical response of a nontarget lesion. The duration of overall CR is measured from the time measurement criteria are first met for CR until the first date that progressive disease is objectively documented, or death due to any cause. Participants without events reported are censored at the last disease evaluation.

As used herein, the term “stable disease” or “SD” means neither sufficient shrinkage to qualify for PR nor sufficient increase to qualify for progressive disease (PD), taking as reference the smallest sum diameters while on study. Duration of stable disease is measured from the start of the treatment until the criteria for progression are met, taking as reference the smallest measurements recorded since the treatment started, including the baseline measurements.

As used herein, the term “progressive disease” or “PD” when refers to a target lesion means at least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). When progressive disease or PD refers to a non-target lesion, it means the appearance of one or more new lesions and/or unequivocal progression of existing non-target lesions. Unequivocal progression should not normally trump target lesion status. It must be representative of overall disease status change, not a single lesion increase.

As used herein, the term “overall survival” or “OS” means the time from randomization (or registration) to death due to any cause. Participants survived are censored on date last known alive.

As used herein, the term “progression-free survival” or “PFS” means the time from randomization (or registration) to the earlier of progression or death due to any cause. Participants alive without disease progression are censored on date of last disease evaluation.

As used herein, the term “best overall response” means the best response recorded from the start of the treatment until disease progression/recurrence (taking as reference for progressive disease the smallest measurements recorded since the treatment started). The patient's best response assignment depends on the achievement of both measurement and confirmation criteria. The duration of an overall response is measured from the time measurement criteria are met for CR or PR (whichever is first recorded) until the first date that recurrent or progressive disease is objectively documented (taking as reference for progressive disease the smallest measurements recorded since the treatment started, or death due to any cause. Participants without events reported are censored at the last disease evaluation).

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate includes hydrate, hemi-hydrate, channel hydrate etc. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treated,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “expose,” “exposed,” “exposure,” or exposing” are used synonymously or interchangeably with the term “treat,” “treated,” “treatment,” or “treating.”

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, each of the terms “partial response” and “partial remission” can refer to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone Marrow transplantation, and immunotherapy. The size of a tumor can be detected by clinical or by radiological means. Tumor-indicating markers can be detected by means well known to those of skill, e.g., ELISA or other antibody-based tests.

As used herein, each of the terms “complete response” or “complete remission” means that a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence and/or size of a tumor can be detected by clinical or by radiological means. Tumor-indicating markers can be detected by means well known to those of skill, e.g., ELISA or other antibody-based tests. A “complete response” does not necessarily indicate that the cancer has been cured, however, as a complete response can be followed by a relapse.

Disclosed herein includes a method of treating a metastatic colorectal cancer. In some embodiments, the method can comprise administering to a metastatic colorectal cancer patient an effective amount of onvansertib and an effective amount of bevacizumab, wherein the patient has not received any treatment comprising bevacizumab within at least three months prior to the administration of onvansertib and bevacizumab.

### Cancer

The methods, compositions and kits disclosed herein can be used to treat various types of cancer. The cancer can be a solid tumor, a liquid tumor, or a combination thereof. In some embodiments, the cancer is a solid tumor, including but not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. In some embodiments, the cancer is a hematological cancer. Non-limiting examples of hematological cancer include Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), and Multiple myeloma (“MM”). Additionally, the disease or condition provided herein includes refractory or recurrent malignancies whose growth may be inhibited using the methods and compositions disclosed herein.

In some embodiments, the cancer is a metastatic cancer. As used herein, “metastatic cancer” can refer to when a cancer spreads (metastasizes) from its original site to another area of the body. Virtually all cancers have the potential to spread this way. Whether metastases develop depends on the complex interaction of many tumor cell factors, including the type of cancer, the degree of maturity (differentiation) of the tumor cells, the location and how long the cancer has been present, as well as other incompletely understood factors. As used herein, the term “metastasis” can refer to formation of progressively growing secondary tumor foci at sites discontinuous from the primary lesion. The metastatic process is a multi-step mechanism in which a metastatic cancer cell escapes from the primary tumor, enters the circulation, invades a distant tissue site and grows into a macroscopic tumor at the target site. The metastatic cancer can be metastatic colorectal cancer, metastatic bladder cancer, metastatic breast cancer, metastatic kidney cancer, metastatic lung cancer, metastatic ovarian cancer, metastatic pancreatic cancer, metastatic prostate cancer, metastatic stomach cancer, metastatic thyroid cancer, metastatic uterine cancer, metastatic renal cancer, metastatic cervical cancer, metastatic recurrent glioblastoma, or a combination thereof.

In some embodiments, the cancer is a metastatic colorectal cancer. In some embodiments, the metastatic colorectal cancer has one or more mutations in one or more RAS genes. RAS genes are a family of genes that encode proteins that regulate cell growth and death through cell signaling pathways. RAS mutations are typically associated with uncontrolled cell proliferation, motility, and protection from apoptosis, and lead to DNA mutation enhancement and other anomalies. The cancer can have one or more mutations in KRAS, NRAS, HRAS, or a combination thereof. In some embodiments, the cancer is KRAS-mutated metastatic colorectal cancer, a NRAS-mutated metastatic colorectal cancer, a HRAS-mutated metastatic colorectal cancer, or a combination thereof.

### PLK1 Inhibitors

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. PLK1 has been shown to be overexpressed in solid tumors and hematologic malignancies, including AML. PLK1 inhibition induces G2-M-phase arrest with subsequent apoptosis in cancer cells, and has emerged as a promising targeted therapy. Several PLK inhibitors have been studied in clinical trials. In a randomized phase II study of patients with AML who were treatment naïve yet unsuitable for induction therapy, the pan-PLK inhibitor, volasertib (BI6727), administered intravenously in combination with LDAC showed a significant increase in OS when compared with LDAC alone. A subsequent randomized phase III study identified no benefit of the combination and described an increased risk of severe infections. PLK1 facilitates HR during Double Strand DNA Break (DSB) Repair. PLK1 phosphorylates Rad51 and BRCA1, facilitating their recruitment to DSB sites and thereby HR-mediated DNA repair. The PLK1 inhibitor can be selective and/or specific for PLK1.

The PLK1 inhibitor can be a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. The PLK1 inhibitor can be onvansertib, BI2536, Volasertib (BI 6727), GSK461364, AZD1775, CYC140, HMN-176, HMN-214, rigosertib (ON-01910), MLN0905, TKM-080301, TAK-960, or Ro3280.

Onvansertib (also known as PCM-075, NMS-1286937, NMS-937, “compound of formula (I)” in U.S. Pat. No. 8,927,530, IUPAC name 1-(2-hydroxyethyl)-8-{[5-(4-methylpiperazin-1-yl)-2-(trifluoromethoxy) phenyl]amino}-4,5-dihydro-1H-pyrazolo[4,3-h]quinazoline-3-carboxamide) is a selective ATP-competitive PLK1 inhibitor. Biochemical assays demonstrated high specificity of onvansertib for PLK1 among a panel of 296 kinases, including other PLK members. Onvansertib has potent in vitro and in vivo antitumor activity in models of both solid and hematologic malignancies. Onvansertib is the first PLK1 specific ATP competitive inhibitor administered by oral route to enter clinical trials with proven antitumor activity in different preclinical models. Onvansertib inhibited cell proliferation at nanomolar concentrations in AML cell lines and tumor growth in xenograft models of AML. Onvansertib also significantly increased cytarabine antitumor activity in disseminated models of AML.

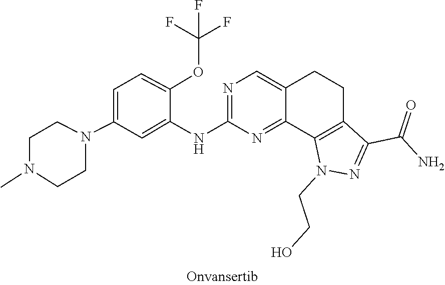 [SMILES: CN1CCN(c2ccc(OC(F)(F)F)c(Nc3ncc4c(n3)-c3c(c(C(N)=O)nn3CCO)CC4)c2)CC1]

Onvansertib shows high potency in proliferation assays having low nanomolar activity on a large number of cell lines, both from solid as well as hematologic tumors. Onvansertib potently causes a mitotic cell-cycle arrest followed by apoptosis in cancer cell lines and inhibits xenograft tumor growth with a clear PLK1-related mechanism of action at well tolerated doses in mice after oral administration. In addition, onvansertib shows activity in combination therapy with approved cytotoxic drugs, such as irinotecan, in which there is enhanced tumor regression in HT29 human colon adenocarcinoma xenografts compared to each agent alone, and shows prolonged survival of animals in a disseminated model of AML in combination therapy with cytarabine. Onvansertib has favorable pharmacologic parameters and good oral bioavailability in rodent and nonrodent species, as well as proven antitumor activity in different nonclinical models using a variety of dosing regimens, which may potentially provide a high degree of flexibility in dosing schedules, warranting investigation in clinical settings. Onvansertib has several advantages over volasertib (BI6727, another PLK1 inhibitor), including a higher degree of potency and specificity for the PLK1 isozyme, and oral bioavailability.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in WO2021/146322, the content of which is incorporated herein by reference in its entirety.

The cancer treatment of the present disclosure can comprise administration of a PLK1 inhibitor (e.g., onvansertib) to a subject with cancer for a desired duration in a cycle, two cycles, or more cycles. The desired duration in each cycle can independently be one, two, three, four, five, six, seven, eight, nine, ten, or more days. The cycle can be, for example, at least 20 days, 21 days, 22 days, 23 days, 24 days, 25 days, 26 days, 27 days, 28 days, 29 days, 30 days, 31 days, 32 days, 33 days, 34 days, 35 days, or more, in length. For example, a single cycle of the treatment can comprise administration of the PLK1 inhibitor (e.g., onvansertib) for four days, five days, six days, seven days, eight days, nine days, ten days, eleven days, twelve days, thirteen days, fourteen days, fifteen days, sixteen days, seventeen days, eighteen days, nineteen days, twenty days, or more in a cycle (e.g., a cycle of at least 21 days (e.g., 21 to 28 days)). In some embodiments, the treatment can comprise administration of the PLK1 inhibitor (e.g., onvansertib) for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) in a single cycle of the treatment can be continuous or with one or more intervals (e.g., one day or two days of break). In some embodiments, the treatment comprises administration of the PLK1 inhibitor (e.g., onvansertib) for five days, ten days, fifteen days, 20 days, or more in a cycle of 21 to 28 days. In some embodiments, the duration of administration of the PLK1 inhibitor in one cycle can be different from the duration of the administration of the PLK1 inhibitor in one or more other cycles. For example, the PLK1 inhibitor can be administered to the subject for 10 days (e.g., day 1 to day 5 in the first 14 days and day 1 to day 5 in the last 14 days in a 28-day cycle) for the first cycle, and for 14 days in the second cycle (e.g., day 1 to day 7 in the first 14 days and day 1 to day 7 in the last 14 days in a 28-day cycle). The length of each of the cycles can vary. For example, cycle 1 can be 28 days, and cycle 2 can be 21 days. In some embodiments, the PLK1 inhibitor (e.g., onvansertib) is administered to the subject five times a week (e.g., Days 1 to 5 of the week), six times a week (e.g., Days 1-6 of the week), or daily.

The cancer treatment disclosed herein can comprise administration of the PLK1 inhibitor (e.g., onvansertib) at, or at about, 12 mg/m²-90 mg/m², for example, as a daily dose. For example, the treatment can comprise daily administration of the PLK1 inhibitor (e.g., onvansertib) at, or at about, 8 mg/m², 10 mg/m², 12 mg/m², 14 mg/m², 15 mg/m², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment (e.g., the first cycle, the second cycle, the third cycle, and a subsequent cycle), or during a single cycle (e.g., the first week, the second week and a subsequent week) of the treatment, for the subject. In some embodiments, the daily dose of the PLK1 inhibitor (e.g., onvansertib) is 12 mg/m², 15 mg/m², 18 mg/m², or 24 mg/m². In some embodiments, the daily dose of the PLK1 inhibitor (e.g., onvansertib) is 15 mg/m². The daily dose of the PLK1 inhibitor (e.g., onvansertib) for each cycle of treatment can vary. For example, the daily dose of the PLK1 inhibitor (e.g., onvansertib) for the first cycle can be 12 mg/m², and the daily dose of the PLK1 inhibitor (e.g., onvansertib) for the second cycle can be increased to, for example, 15 mg/m². In some embodiments, the daily dose of the PLK1 inhibitor (e.g., onvansertib) for the second cycle can then be increased to, for example, 18 mg/m². Without being bound by any particular theory, it is believed that the mg/m² doses disclosed herein are Body Surface Area (BSA) based doses corresponding to flat doses in the range of 20 to 45 mg.

The PLK1 inhibitor (e.g., onvansertib) can also be administered to the subject at a daily dose of about 5-80 mg, for example 5 mg, 10 mg, 15 mg, 20 mg, 25 mg, 30 mg, 35 mg, 40 mg, 45 mg, or 50 mg. The daily dose of the PLK1 inhibitor (e.g., onvansertib) can be adjusted (e.g., increased or decreased with the range) during the treatment (e.g., the first cycle, the second cycle, the third cycle, and a subsequent cycle) or during a single cycle (e.g., the first week, the second week and a subsequent week) of the treatment, for the subject. For example, the subject can be administered with 30 mg onvansertib on the first day or the first several days, with the option to decrease the dose to 25 mg, 20 mg, 15 mg, or 10 mg.

A maximum concentration (Cₘₐₓ) of the PLK1 inhibitor (e.g., onvansertib) in a blood of the subject (during the treatment or after the treatment) when the PLK1 inhibitor is administered alone or in combination with one or more additional cancer therapeutics (e.g., FOLFIRI and bevacizumab) can be from about 100 nmol/L to about 1500 nmol/L. For example, the Cₘₐₓ of the PLK1 inhibitor (e.g., onvansertib) in a blood of the subject when the PLK1 inhibitor is administered alone or in combination with one or more additional cancer therapeutics (e.g., FOLFIRI and bevacizumab) can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with one or more additional cancer therapeutics (e.g., FOLFIRI and bevacizumab) can be from about 1000 nmol/L·hour to about 400000 nmol/L·hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with one or more additional cancer therapeutics (e.g., FOLFIRI and bevacizumab) can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of the PLK1 inhibitor (e.g., onvansertib) in a blood of the subject when the PLK1 inhibitor is administered alone or in combination with one or more additional cancer therapeutics (e.g., FOLFIRI and bevacizumab) can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the one or more additional cancer therapeutics (e.g., FOLFIRI and bevacizumab) can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with one or more additional cancer therapeutics (e.g., FOLFIRI and bevacizumab) can be from about 10 hours to about 60 hours. For example, the elimination half-life (T_{1/2}) of the PLK1 inhibitor (e.g., onvansertib) in a blood of the subject when the PLK1 inhibitor is administered alone or in combination with one or more additional cancer therapeutics (e.g., FOLFIRI and bevacizumab) can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

### Anti-Angiogenics

Angiogenesis is known as the inappropriate formation of new blood vessels, and is typically required for cancer metastasis. “Angiogenesis” often occurs in tumors when endothelial cells secrete a group of growth factors that are mitogenic for endothelium causing the elongation and proliferation of endothelial cells which results in a generation of new blood vessels. The inhibition of angiogenesis can cause tumor regression in animal models and has also been found to be effective in humans for treatment of metastatic cancer. The terms “anti-angiogenics” and “angiogenesis inhibitor” shall have their ordinary meaning, and can also be used interchangeably herein to refer to any agent that can inhibit angiogenesis. Several anti-angiogenics have been approved for use in humans for the treatment of cancer. In some embodiments of the methods disclosed herein, the subject has not received any prior treatment comprising administration of an angiogenesis inhibitor.

The angiogenesis inhibitor and/or the anti-angiogenics can be capable of inhibiting VEGF-A, VEGFR-1, VEGFR-2, VEGFR-3, EGFR, HER2, PDGFR family proteins, RAF, Kit (or c-Kit), FLT3, CSF-1R, RET, Abl, Itk, LcK, c-FMS, FGFR family proteins, c-Met, P1GF, TNF-α, IFNs, ILs, bFGF, mTOR, or any combination thereof. The anti-angiogenics can be Afatinib (Gilotrif®), Axitinib (Inlyta®), bevacizumab (Avastin®), Cabozantinib (Cometriq®), Cetuximab (Erbitux®), Erlotinib (Tarceva®), Everolimus (Afinitor®), Gefitinib (Iressa®), Imatinib (Gleevec®), Lapatinib (Tykerb®), Lenalidomide (Revlimid®), Lenvatinib mesylate (Lenvima®), Necitumumab (Portrazza™), Neratinib (Nerlynx®), Panitumumab (Vectibix®), Pazopanib (Votrient®), Pertuzumab (Perjeta®), Ramucirumab (Cyramza®), Regorafenib (Stivarga®), Sorafenib (Nexavar®), Sunitinib (Sutent®), Thalidomide (Synovir, Thalomid®), Trastuzumab (Ontruzant®), Vandetanib (Caprelsa®), or Ziv-aflibercept (Zaltrap®). The angiogenesis inhibitor can be Afatinib (Gilotrif®), Axitinib (Inlyta®), bevacizumab (Avastin®), Cabozantinib (Cometriq®), Cetuximab (Erbitux®), Erlotinib (Tarceva®), Everolimus (Afinitor®), Gefitinib (Iressa®), Imatinib (Gleevec®), Lapatinib (Tykerb®), Lenalidomide (Revlimid®), Lenvatinib mesylate (Lenvima®), Necitumumab (Portrazza™), Neratinib (Nerlynx®), Panitumumab (Vectibix®), Pazopanib (Votrient®), Pertuzumab (Perjeta®), Ramucirumab (Cyramza®), Regorafenib (Stivarga®), Sorafenib (Nexavar®), Sunitinib (Sutent®), Thalidomide (Synovir, Thalomid®), Trastuzumab (Ontruzant®), Vandetanib (Caprelsa®), or Ziv-aflibercept (Zaltrap®). In some embodiments, the anti-angiogenics is bevacizumab. Bevacizumab (also referred to herein as “bev”) can be used in combination with chemotherapy in both 1^{st} and 2^{nd} lines of therapy for treating cancer. Other anti-angiogenics, e.g., ramucirumab and aflibercept, can be used in 2^{nd} line setting. Previously, overall survival (OS) and median progression free survival (mPFS) benefit in 2^{nd} line have been shown to be independent of whether bevacizumab was given in 1^{st} line (e.g., the subject received prior treatment comprising inhibiting angiogenesis). For example, in studies examining OS and PFS in patients with and without prior bevacizumab treatment, it was found that OS was 13.9 months without prior bevacizumab vs. 12.5 months with prior bevacizumab. For PFS, mPFS was 6.9 months without prior bevacizumab vs. 6.7 months with prior Bevacizumab. Anti-angiogenic therapies incrementally improve response rates in patients with prior bevacizumab vs without prior bevacizumab. The objective response rate of patients who had received prior bevacizumab is 5% to 13% as compared to ˜25% with no prior bevacizumab.

### Combination Treatment for Cancer

Provided herein include a combination therapy of an anti-angiogenics (e.g., bevacizumab) and a PLK1 inhibitor (e.g., onvansertib) and related methods, compositions, and kits used for treating metastatic cancer, including mCRC. In some embodiments, a method for treating cancer comprises administrating a combination of an anti-angiogenics (e.g., bevacizumab) and a PLK1 inhibitor (e.g., onvansertib), or a pharmaceutically acceptable salt, solvate, stereoisomer, or prodrug thereof, to a subject in need thereof (e.g., a subject with metastatic cancer) who has not received any treatment comprising bevacizumab within at least three months prior to the combination therapy. In some embodiments, the subject has not received any treatment comprising bevacizumab within about, at least, or at least about 3 months prior to the administration of the anti-angiogenics (e.g., bevacizumab) and the PLK1 inhibitor (e.g., onvansertib). For example, the subject has not received any prior bevacizumab treatment within about 3 months, 3.5 months, 4 months, 4.5 months, 5 months, 5.5 months, 6 months, 7 months, 8 months, 9 months, 10 months, 11 months, 1 year, 2 years, 3 years, 4 years, 5 years, 6 years, 7 years, 8 years, 9 years, 10 years, or a number or a range between any two of these values, prior to the administration of the anti-angiogenics (e.g., bevacizumab) and the PLK1 inhibitor (e.g., onvansertib). The subject can, for example, be a subject who has prior cancer diagnosis. The subject can, for example, be a subject who has no prior cancer diagnosis. In some embodiments, the subject has not received any treatment comprising bevacizumab within about 3, 3.1, 3.2, 3.3, 3.4, 3.5, or 4 months prior to the combination treatment. In some embodiments, the subject is known to have not been exposed to bevacizumab for about, at least, or at least about three months (e.g., at least 3 months, 4 months, 5 months, 6 months, 7 months, 8 months, 9 months, 10 months, 11 months, 1 year, 2 years, 3 years, 4 year, 5 years, 6 years, 7 years, 8 years, 9 years, 10 years or a number or a range between any two of these values, prior to the administration of the PLK1 inhibitor (e.g., onvansertib) and the anti-angiogenics (e.g., bevacizumab). The method comprises administrating an anti-angiogenics and a PLK1 inhibitor to the patient in a manner sufficient to inhibit progression of the cancer. For example, the anti-angiogenics and the PLK1 inhibitor can be administrated to a subject with cancer simultaneously, separately, or sequentially. In some embodiments, the anti-angiogenics is bevacizumab and the PLK1 inhibitor is onvansertib. The subject can be a human patient suffering from metastatic cancer such as metastatic colorectal cancer, including KRAS-mutated metastatic colorectal cancer, HRAS-mutated metastatic colorectal cancer, and/or NRAS-mutated metastatic colorectal cancer. In some embodiments, the subject has not received any treatment comprising bevacizumab within a determined time period prior to the combination therapy, where the determined time period is at least 3 months, 3.5 months, 4 months, 4.5 months, 5 months, 5.5 months, 6 months, 7 months, 8 months, 9 months, 10 months, 11 months, 1 year, 2 years, 3 years, or a number or a range between any two of these values. The prior bevacizumab treatment can be a bevacizumab treatment for the metastatic cancer (e.g., mCRC).

In some embodiments, the subject has received at least one prior treatment comprising the anti-angiogenics (e.g., bevacizumab) prior to the combination treatment with the anti-angiogenics (e.g., bevacizumab) and the PLK1 inhibitor (e.g., onvansertib), but the prior bevacizumab-containing treatment(s) had ended at least 3 months prior to the combination treatment with the anti-angiogenics (e.g., bevacizumab) and the PLK1 inhibitor (e.g., onvansertib). The subject has not been exposed to bevacizumab since then until the combination treatment with the anti-angiogenics (e.g., bevacizumab) and the PLK1 inhibitor (e.g., onvansertib). Thus, the subject has not been exposed for bevacizumab for at least 3 months (e.g., 3 months, 4 months, 5 months, or longer). In some embodiments, the subject has not received any bevacizumab treatment within about 3 months prior to the combination treatment. The subject may have received at least one prior bevacizumab treatment (e.g., one, two, or even more bevacizumab treatments) more than 3 months prior to the combination therapy.

The present disclosure has demonstrated that a combination treatment with an anti-angiogenics (e.g., bevacizumab) and a PLK1 inhibitor (e.g., onvansertib) can surprisingly result in significantly enhanced efficacy against metastatic cancer in a subject who has not received any prior treatment comprising anti-angiogenesis (e.g., bevacizumab) within about 3 months, especially when compared to subjects who have received a prior treatment comprising anti-angiogenesis (e.g., bevacizumab) within about 3 months (e.g., within about 2 months or within about one month). The subject who has not received any prior treatment comprising bevacizumab within about 3 months can achieve highly improved tumor inhibition rate, higher objective response rate (ORR) and/or higher progression free survival (PFS). In some embodiments, the subject who has not received any prior treatment containing bevacizumab within about 3 months can reduce the number of cancer cells detected in the subject by about, at least, at least about 25%, 30%, 40%, 50%, 60%, or 70% relative to subjects who have been recently exposed to bevacizumab (e.g., within 3 months). In some embodiments, the subject who has not received any prior treatment containing bevacizumab within about 3 months can achieve about, at least, at least about 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500% or a number or a range between any two of these values, higher ORR compared to subjects who have been recently exposed to bevacizumab (e.g., within 3 months). In some embodiments, the subject who has not received any prior treatment containing bevacizumab within about 3 months can achieve about, at least, at least about 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500% or a number or a range between any two of these values, higher PFS compared to subjects who have received a prior treatment containing bevacizumab within about three months (e.g., within 3 months, within 2 months, within 1 month).

Accordingly, the methods, compositions, and kits disclosed herein can be particularly beneficial for patients who have received at least one prior cancer treatment comprising an anti-angiogenics (e.g., bevacizumab). The at least one prior cancer treatment may have failed, stopped working, and/or had side effects that were not tolerated by the subjects. In some embodiments, the subject being treated by the methods disclosed herein has failed a treatment or is intolerant of the treatment comprising bevacizumab (alone or in combination with other cancer treatment). The at least one prior cancer treatment comprising the anti-angiogenics can further comprise other standard of care chemotherapy treatment. For example, other standard chemotherapy treatment can comprise a chemotherapy containing fluorouracil (e.g., FOLFIR or FOLFOX). In some embodiments, standard of care chemotherapy comprises fluoropyrimidine and oxaliplatin. In some embodiments, other standard of care chemotherapy treatment comprises FOLFIRI, abiraterone, FOLFOX, an anti-EGFR agent, a KRAS directed inhibitor, gemcitabine, abraxane, nanoliposomal irinotecan, 5-FU, FOLFIRINOX, FOLFOXIRI, or a combination thereof. In some embodiments, the prior bevacizumab treatment has been administered to the patient in combination with FOLFIR or FOLFOX.

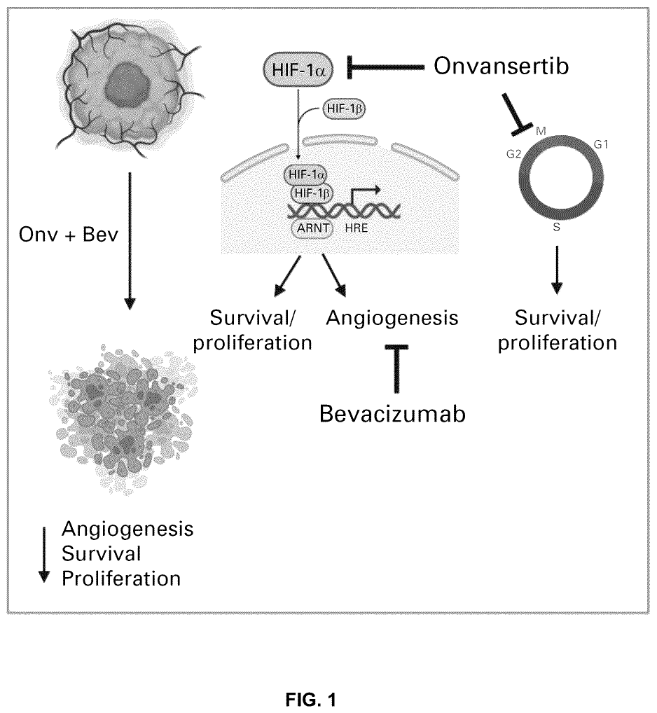

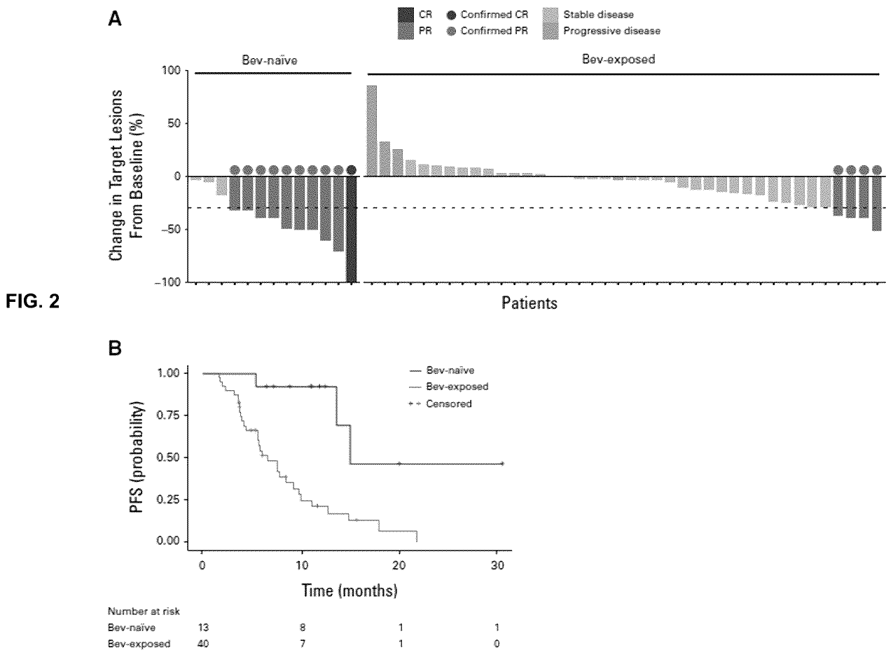

Data from clinical trial NCT03829410 showed that bevacizumab-exposed patients are less sensitive to onvansertib combined with FOLFIRI+bevacizumab compared to bevacizumab-naïve patients (see FIG. 1 in Ahn, Daniel H., et al. “Onvansertib in combination with chemotherapy and bevacizumab in second-line treatment of KRAS-mutant metastatic colorectal cancer: A single-arm, phase II trial.” Journal of Clinical Oncology (2024): JCO-24, the content of which is incorporated herein by reference), suggesting that bevacizumab may confer resistance to the combination. The translational research supports that bevacizumab exposure may result in transcriptional changes that contribute to resistance to both onvansertib and bevacizumab as shown in FIG. 2.

Surprisingly, however, the present disclosure has found that the resistance associated with prior bevacizumab treatment can be reverted in tumors not exposed to bevacizumab for a certain period of time (e.g., three months or longer). In Bev-exposed tumors, Bev exposure leads to upregulation of mitotic and hypoxia pathways, including the oncogenic signature related to the angiogenic factor placental growth factor (PGF), resulting in resistance to both Onv and Bev. Without being bound by any theory, it is believed that such upregulation of mitotic and hypoxia pathways can be reverted following a certain bevacizumab-free period (e.g., at least three months). In some embodiments, the methods, compositions and kits described herein can be used as a second line cancer treatment for metastatic cancer such as metastatic colorectal cancer.

In some embodiments, the subject has not received any treatment comprising bevacizumab within about three months prior to the administration of onvansertib and bevacizumab. In some embodiments, the subject is known to have not received any treatment comprising bevacizumab within about three months prior to the administration of onvansertib and bevacizumab. In some embodiments, the subject has been identified as having not received any treatment comprising bevacizumab within about three months prior to the administration of onvansertib and bevacizumab. In some embodiments, the patient has received a prior treatment comprising bevacizumab, and the prior treatment ended at least 3 months (e.g., 3 months, 4 months, 5 months, 6 months, 7 months, 8 months, 9 months, 10 months, 11 months, 1 year, 2 years, or longer) prior to the combination treatment.

In some embodiments, the patient has received at least one prior treatment not comprising bevacizumab prior to the combination therapy. The at least one prior treatment not comprising bevacizumab can be performed within about 3 months or more than about 3 months prior to the combination therapy. The at least one prior treatment can comprise other chemotherapy agents such as fluoropyrimidine, oxaliplatin, irinotecan and anti-epidermal growth factor agents such as cetuximab and panitumumab and others treatment modalities for metastatic colorectal cancer identifiable to a person skilled in the art.

In some embodiments, the method described herein can further comprise at least one additional treatment comprising bevacizumab to the patient, following the combination treatment with onvansertib and bevacizumab. The additional bevacizumab treatment can further comprises another chemotherapy agent, such as a PLK1 inhibitor (e.g., onvansertib), FOLFOX, FOLFIRI, 5-fluorouuracil (5-FU), FOLFIRINOX, FOLFOXIRI, abiraterone, an anti-EGFR agent, a KRAS-directed inhibitor, gemcitabine, abraxane, nanoliposomal irinotecan, or a combination thereof. In some embodiments, the additional bevacizumab treatment further comprises onvansertib. In some embodiments, the additional bevacizumab treatment further comprises FOLFOX and/or FOLFIRI. In some embodiments, the additional bevacizumab treatment comprises bevacizumab, onvansertib and FOLFOX/FOLFIRI. The additional treatment can begin at least three months (e.g., 3 months, 4 months, 5 months, 6 months, or 1 year) after the combination treatment with onvansertib and bevacizumab has ended. Additional treatments can be conducted to the subject as needed to achieve desired therapeutic effects. In some embodiments, two consecutive bevacizumab-containing treatments are separated by a time period of at least 3 months (e.g., 3 months, 4 months, 5 months, 6 months, 1 year or longer) such that the resistance associated with bevacizumab treatment can be reverted.

The method for treating cancer can, in some embodiments, comprises identifying a subject having a metastatic colorectal cancer and has not received any treatment comprising bevacizumab within about three months prior to the administration of onvansertib and bevacizumab; and administering a PLK1 inhibitor and an anti-angiogenics to the subject, thereby reducing or inhibiting progression of the metastatic colorectal cancer. The method, in some embodiments, comprises identifying a subject having a metastatic cancer and has not been exposed to bevacizumab for at least three months prior to the administration of onvansertib and bevacizumab; and administering a PLK1 inhibitor and an anti-angiogenics to the subject, thereby reducing or inhibiting progression of the metastatic cancer.

In some embodiments, the method comprises excluding patients who have received a prior treatment comprising bevacizumab within about three months from receiving the combination treatment. For example, if the patient has been identified or is known as having recently received a treatment comprising bevacizumab within three months, two months, or one month, the patient will be declined from receiving the combination treatment.

In some embodiments, administering the PLK1 inhibitor and the anti-angiogenics can surprisingly result in significantly enhanced efficacy against metastatic cancer in a subject that has not received any prior treatment comprising anti-angiogenesis (e.g., bevacizumab) within three months, causing tumor regression and cancer survival. In some embodiments, administering the PLK1 inhibitor and the anti-angiogenics synergistically improves the ORR and/or the PFS of the subjects relative to the PLK1 inhibitor treatment alone, the anti-angiogenics treatment alone, and/or the additive effect of the PLK1 inhibitor treatment alone and the anti-angiogenics treatment alone. In some embodiments, administering the PLK1 inhibitor and the anti-angiogenics improves one or more therapeutic effects in the subject relative to a control or a baseline.

In some embodiments, administering the PLK1 inhibitor and the anti-angiogenics synergistically reduces or inhibits progression of the metastatic cancer relative to the PLK1 inhibitor treatment alone, the anti-angiogenics treatment alone, and/or the additive effect of the PLK1 inhibitor treatment alone and the anti-angiogenics treatment alone.

The inhibition or reduction of cancer progression that can be achieved by the methods disclosed herein using the PLK1 inhibitor (e.g., onvansertib) and the anti-angiogenics (e.g., bevacizumab) is not merely additive, but is enhanced or synergistic (that is, the inhibition is greater than the combined inhibition of progression caused by the anti-angiogenics alone plus the PLK1 inhibitor alone). The enhanced or synergistic efficacy or inhibition of any combination of an anti-angiogenics and a PLK1 inhibitor of the present disclosure can be different in different embodiments. In some embodiments, the enhanced or synergistic efficacy or inhibition of any combination of an anti-angiogenics and a PLK1 inhibitor of the present disclosure is, is about, is at least, is at least about, is at most, or is at most about, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values, higher than the combined inhibition of progression caused by the anti-angiogenics alone plus the PLK1 inhibitor alone.

The molar ratio of the PLK1 inhibitor (e.g., onvansertib) to the anti-angiogenics (e.g., bevacizumab) can be, for example, about 1:200, 1:100, 1:90, 1:80, 1:70, 1:60, 1:50, 1:40, 1:30, 1:20, 1:10, 1:1, 10:1, 20:1, 30:1, 40:1, 50:1, 100:1, 1000:1, 2000:1, or 5000:1, or a number or a range between any two of these values. In some embodiments, the enhanced or synergistic efficacy or inhibition of cancer progression caused by a combination of the anti-angiogenics and the PLK1 inhibitor (e.g., onvansertib) is, is about, is at least, is at least about, is at most, or is at most about 20%, 30%, 40%, 50%, 60%, 70%, 80%, 90%, 100%, 110%, 120%, 130%, 140%, 150%, 160%, 170%, 180%, 190%, 200%, 250%, 300%, or a number or a range between any two of these values, higher than the combined inhibition of progression caused by the anti-angiogenics alone plus the PLK1 inhibitor (e.g., onvansertib) alone. For example, a combination of the anti-angiogenics and the PLK1 inhibitor can cause a 50%, 60%, 70%, 80%, 90%, or more, inhibition of cancer progression (cancer cell viability of 50%, 40%, 30%, 20%, 10%, or less), whereas under the same conditions the combined inhibition of the anti-angiogenics alone plus the PLK1 inhibitor alone can be 10%, 20%, 25%, 30%, or less) inhibition of cancer progression (cancer cell viability of 90%, 80%, 75%, 70%, or more). Thus, the enhanced or synergistic efficacy or inhibition of cancer progression caused by the combination of the anti-angiogenics and the PLK1 inhibitor for example, 50%, 60%, 70%, 80%, 90%, 100%, or more higher than the combined inhibition of progression caused by the anti-angiogenics alone plus the PLK1 inhibitor alone. In some embodiments, the anti-angiogenics is bevacizumab and the PLK1 inhibitor is onvansertib.

The anti-angiogenics and the PLK1 inhibitor can be administered to the patient in any manner deemed effective to treat the cancer. The anti-angiogenics can be administered together with, or separately from, the PLK1 inhibitor. When administered separately, the anti-angiogenics can be administered before or after the PLK1 inhibitor, or in different administration cycles. The administration of the PLK1 inhibitor can be oral administration. The administration of the anti-angiogenics can be intravenous administration or oral administration.

The PLK1 inhibitor and the anti-angiogenics can be administered simultaneously or sequentially. In some embodiments, it can be advantageous to administer the PLK1 inhibitor (e.g., onvansertib) to the subject before the anti-angiogenics (e.g., bevacizumab), e.g., on one or more days, or each day, of the days on which the PLK1 inhibitor and the anti-angiogenics are administered to the subject. The time interval between the administration of the PLK1 inhibitor and the administration of the anti-angiogenics can be, for example, 30 minutes, 1 hour, 2 hours, 3 hours, 4 hours, 5 hours, 6 hours, 7 hours, 8 hours, 9 hours, 10 hours, 11 hours, 12 hours, a range between any two of these values, or any value between 30 minutes and 12 hours. In some embodiments, the PLK1 inhibitor (e.g., onvansertib) and the anti-angiogenics (e.g., bevacizumab) are both administered to the subject on, or on at least about, 50%, 60%, 70%, 75%, 80%, 85%, 90%, 95%, or 100% of the days in a cycle (e.g., in each cycle during the combination treatment), and optionally the PLK1 inhibitor is administered to the subject prior to the anti-angiogenics on each of the days both are administered, for example the PLK1 inhibitor is administered 30 minutes, 1 hour, 2 hours, 3 hours, 4 hours, 5 hours, 6 hours, 7 hours, 8 hours, 9 hours, 10 hours, 11 hours, 12 hours, a range between any two of these values, or any value between 30 minutes and 12 hours, prior to the administration of the anti-angiogenics.

The anti-angiogenics and the PLK1 inhibitor can each be administered in any schedule, e.g., once or multiple times per day or week; once, twice, three times, four times, five times, six times or seven times (daily) per week; for one or multiple weeks; etc. In some embodiments, the anti-angiogenics and the PLK1 inhibitor are each administered to the patient in a cycle of at least twice within a week. In other embodiments, the anti-angiogenics and the PLK1 inhibitor are each administered to the patient in a cycle of at least five times within a week. In some embodiments, the PLK1 inhibitor is administered daily, and the anti-angiogenics are administered daily, weekly, bi-weekly, every four weeks, every five weeks, or monthly. In further embodiments, the patient undergoes at least two cycles of administration. The patient can undergo one cycle or more than one cycle of administrations, for example, two cycles, three cycles, three cycles, four cycles, five cycles, or more. Two adjacent cycles of administration can be continuous, i.e., no break between the last day of the first cycle and the first day of the second cycle. In some embodiments, two adjacent cycles of administration have a break between them, i.e., an interval between the last day of the first cycle and the first day of the second cycle. The break (i.e., the interval) can be or be at least, one day, two days, three days, five days, seven days, ten days, two weeks, three weeks, four weeks, one month, two months, three months, four months, five months, six months, or a number or a range between any two of these values. In some embodiments, the patient undergoes three or four cycles of administration in which each cycle comprises at least five times within a week (e.g., 5 days per week). Each of the cycle in a multi-cycle administration can have the same dosing schedule, or different. For example, one of the cycle in the multi-cycle administration can be five continuous days of daily administration of the PLK1 inhibitor and anti-angiogenics and two days of break in one week for four weeks, and one or more other cycles in the same multi-cycle administration be 28 continuous days of daily administration of the PLK1 inhibitor and the anti-angiogenics in a four-week period. The PLK1 inhibitor can be administered on at least four days in the cycle. In some embodiments, PLK1 inhibitor is not administered on at least one day in the cycle.

The anti-angiogenics can be administered to the patient at any appropriate dosage, e.g., a dosage of about, at least or at most 0.1 mg/kg, 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, 1500 mg/kg, 2000 mg/kg, or a number between any two of these values. The dosage unit based on the body weight (mg/kg) can be converted to another unit (e.g., mg/m²) using a conversion chart such as the body surface area (BSA) conversion chart as will be understood by a person of skill in the art. The anti-angiogenics (e.g., bevacizumab) can be administered at about 1 mg/kg-20 mg/kg. In some embodiments, the anti-angiogenics is bevacizumab, which is administered at a dosage of about, at least or at most 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, or a number between any two of these values. Bevacizumab can be administered at about 5 mg/kg, about 7.5 mg/kg, about 10 mg/kg, or about 15 mg/kg.

The anti-angiogenics can be administrated to the patient once daily, twice daily, or three times daily. The anti-angiogenics can be administered daily, weekly, bi-weekly, every three weeks, every four weeks, or every month. In some embodiments, the anti-angiogenics is administered in a cycle of 7-56 days of daily, weekly, bi-weekly, tri-weekly, every four weeks, or monthly. In some embodiments, the anti-angiogenics is administered in a cycle of 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 32 days, 35 days, 42 days, 49 days, or 56 days. In some embodiments, the anti-angiogenics is administered in 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 32 days, 35 days, 42 days, 49 days, or 56 days, in a cycle. In some embodiments, the anti-angiogenics is administered in day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, day 35, day 36, day 37, day 38, day 39, day 40, day 41, day 42, day 43, day 44, day 45, day 46, day 47, day 48, day 49, day 50, day 51, day 52, day 52, day 53, day 54, day 55, and/or day 56. In some embodiments, the anti-angiogenics is not administered in day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 31, day 32, day 33, day 34, day 35, day 36, day 37, day 38, day 39, day 40, day 41, day 42, day 43, day 44, day 45, day 46, day 47, day 48, day 49, day 50, day 51, day 52, day 52, day 53, day 54, day 55, and/or day 56.

In some embodiments, the subject has not received a treatment comprising an agent that can inhibit angiogenesis (e.g., an anti-angiogenic) within about three months prior to the administration of the PLK1 inhibitor (e.g., onvansertib) and the anti-angiogenics (e.g., bevacizumab). In some embodiments, the patient has not been exposed to an anti-angiogenic agent (e.g., bevacizumab) for at least 3 months. For example, the patient has not been exposed to bevacizumab for 3 months, 4 months, 5 months, 6 months, 7 months, 8 months, 9 months, 10 months, 11 months, 1 year, 2 years, 3 years or longer. The angiogenesis inhibitor can be the same as the anti-angiogenics. Any agent that can inhibit angiogenesis (e.g., an anti-angiogenic), now known or later discovered, can be used in these methods, including anti-angiogenics that inhibit the activity of one or more growth factors or any other protein that promotes angiogenesis. For example, the angiogenesis inhibitor and/or the anti-angiogenics can be capable of inhibiting vascular endothelial growth factor family proteins or the signaling pathways of said proteins such as VEGF-A, VEGFR-1, VEGFR-2, VEGFR-3, and P1GF. Other proteins and pathways that can be targeted by anti-angiogenics include, but are not limited to: epidermal growth factor receptor (EGFR), human epidermal growth factor receptor 2 (HER2), platelet-derived growth factor receptor (PDGFR) family proteins, RAF kinases; tyrosine kinases including Kit (or c-Kit), RET, Abl, Itk, LcK, c-Met, and FLT3; colony stimulating factor 1 receptor (CSF-1R) and c-FMS, fibroblast growth factor receptor (FGFR) family proteins, tumor necrosis factor alpha (TNF-α), interferons (IFNs), interleukins (ILs), basic fibroblast growth factor (bFGF), mammalian target of rapamycin (mTOR), or any combination thereof. The anti-angiogenic can be a small molecule, an antibody or fragment thereof, an aptamer, an RNA (e.g., a miRNA), or any other agent capable of inhibiting angiogenesis. In some embodiments, the anti-angiogenic or angiogenesis inhibitor is bevacizumab (e.g., Avastin®).

Similarly, any PLK1 inhibitor, now known or later discovered, can be used in these methods, including PLK1 inhibitors that are selective for PLK1, and PLK1 inhibitors that also inhibit the activity of other proteins. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or a combination thereof. In some of these embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280.

The PLK1 inhibitor can be onvansertib. In these embodiments, onvansertib is administered to the patient at any appropriate dosage, e.g., a dosage of less than 12 mg/m², less than or equal to 24 mg/m², or greater than 24 mg/m². In some embodiments, onvansertib is administered to the patient at about 12 mg/m², at about 15 mg/m², or at about 18 mg/m². In some embodiments, the onvansertib is administered to the patient daily. In additional embodiments, onvansertib is administered in a cycle of 3-10 days of daily onvansertib administration with 2-16 days with no onvansertib administration. In some embodiments, onvansertib is administered to the patient in a cycle of at least five times within a week. The patient can undergo two, three, or four cycles of administration. In some embodiments, the patient undergoes four cycles of administration in a cycle of at least five days of daily onvansertib administration with 1-2 days with no onvansertib administration.

In some embodiments, a PLK1 inhibitor alone or in combination with an anti-angiogenics is administrated to a patient who has taken a drug holiday after undergoing one or more cycles of administration. A drug holiday as used herein refers to a period of time when a patient stops taking a PLK1 inhibitor and/or an anti-angiogenics. A drug holiday can be a few days to several months. In some embodiments, the drug holiday can be 1 week, 2 weeks, 3 weeks, 4 weeks, 5 weeks, 6 weeks, 7 weeks, 8 weeks, 9 weeks, 10 weeks, or any value or a range between any two of these values.

As can be appreciated by one of skill in the art, the amount of administration of the anti-angiogenics and the PLK1 inhibitor, and the timing of the administration, can depend on the type (species, gender, age, weight, etc.) and condition of the subject being treated and the severity of the disease or condition being treated. The anti-angiogenics and the PLK1 inhibitor can formulated into a single pharmaceutical composition, or two separate pharmaceutical compositions. The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Methods, compositions, kits and systems disclosed herein can be applied to different types of subjects. For example, the subject can be a subject receiving a cancer treatment, a subject at cancer remission, a subject has received one or more cancer treatment, or a subject suspected of having cancer, wherein the subject has not received any treatment comprising bevacizumab within three months prior to the administration of the PLK1 inhibitor (e.g., onvansertib) and the anti-angiogenics (e.g., bevacizumab). In some embodiments, the patient has received a prior cancer treatment. The cancer treatment may or may not comprise an anti-angiogenics. In some embodiments, the patient has received a prior cancer treatment comprising an anti-angiogenics (e.g. bevacizumab) more than 3 months (e.g., 4 months, 5 months, 6 months, 7 months, 8 months, 9 months, 10 months, 11 months, 1 year, 2 years, 3 years, 4 year, 5 years, 6 years, 7 years, 8 years, 9 years, 10 years or a number or a range between any two of these values) prior to the administration of a PLK1 inhibitor (e.g., onvansertib) and an anti-angiogenics (e.g. bevacizumab). The patient has been bev-free (e.g., with no bev exposure) for at least three months. The subject can have a stage I cancer, a stage II cancer, a stage III cancer, and/or a stage IV cancer. In some embodiments, the subject has stage IV cancer. In some embodiments, the subject has metastatic cancer. In some embodiments, the subject has not received any prior treatment comprising inhibiting angiogenesis.

The treatment of the present disclosure can comprise administration of a PLK1 inhibitor (onvansertib) for a desired duration in a cycle. The administration of the PLK1 inhibitor (and/or the anti-angiogenics) can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the PLK1 inhibitor (and/or the anti-angiogenics) is administered to the patient. The administration can be, for example, once every two days, every three days, every four days, every five days, every six days, or every seven days. The length of the desired duration can vary, for example, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the PLK1 inhibitor (e.g., onvansertib) and/or the anti-angiogenics agents for four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). In some embodiments, the treatment can comprise administration of the PLK1 inhibitor (e.g., onvansertib) and/or the anti-angiogenics for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) and/or the anti-angiogenics in a single cycle of the treatment can be continuous or with one or more intervals (e.g., one day or two days of break). In some embodiments, the treatment comprises administration of the PLK1 inhibitor (e.g., onvansertib) for five days in a cycle of 21 to 28 days.

In some embodiments, the PLK1 inhibitor (e.g., onvansertib) is administered to the subject in need thereof on twenty days (e.g., Days 1-10 and 15-24) during a 28-day cycle. The twenty days can be, for example, a continuous daily administration for ten days (e.g., Days 1-10) and another continuous daily administration (e.g., Days 15-24) for ten days, or a continuous daily administration for four sets of five days (e.g., Days 1-5, 8-12, 15-19, and 22-26), In some embodiments, for example when the patient is identified to have low tolerance to the PLK1 inhibitor, the PLK1 inhibitor is administered to the subject in need thereof on ten days (e.g., Days 1-5 and 15-19) during a 28-day cycle. The ten days can be, for example, a continuous daily administration for ten days (e.g., Days 1-10) or two continuous daily admiration for five days each (e.g., Days 1-5 and Days 15-19). In some embodiments, the PLK1 inhibitor is administered to the subject in need thereof daily throughout the whole cycle (e.g., daily for 28 days in a cycle of 28 days). Depending on the needs of inhibition/reversion of cancer progression in the subject, the subject can receive one, two, three, four, five, six, or more cycles of treatment. For combination treatment, the administration cycles, dosing schedules, and/or dosage amounts of the anti-angiogenics and the PLK1 inhibitor can be the same or different. For combination treatment, the administration cycle, dosing schedule, and/or dosage amount of the anti-angiogenics can be adjusted according to the administration cycle, dosing schedule, and/or dosage amount of the PLK1 inhibitor. For example, the anti-angiogenics (e.g., bevacizumab) can be administered in four 7-day cycles (e.g., daily dose on Days 1-5 and no dose on Days 6-7, repeated for 4 weeks), which corresponds to a 28-day cycle for administration of the PLK1 inhibitor (e.g., onvansertib).

The treatment can comprise administration of the PLK1 inhibitor (e.g., onvansertib) at, or at about, 6 mg/m²-90 mg/m², for example, as a daily dose. For example, the treatment can comprise daily administration of the PLK1 inhibitor (e.g., onvansertib) at, or at about, 6 mg/m², 8 mg/m², 10 mg/m², 12 mg/m², 14 mg/m², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a number or a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK1 inhibitor (e.g., onvansertib) is administered at 12 mg/m² on twenty days (e.g., Days 1-10 and 15-24) during a 28-day cycle. In some embodiments, the PLK1 inhibitor (e.g., onvansertib) is administered at 15 mg/m² on ten days (e.g., Days 1-5 and 15-19) during a 28-day cycle. In some embodiments, the PLK1 inhibitor (e.g., onvansertib) is administered at 8 mg/m² or 10 mg/m² everyday (e.g., Days 11-28) during a 28-day cycle. In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK1 inhibitor is administered at or at about 12 mg/m². In some embodiments, the PLK1 inhibitor is administered at or at about 15 mg/m². In some embodiments, the PLK1 inhibitor is administered at or at about 18 mg/m². The PLK1 inhibitor (e.g., onvansertib) can also be administered to the subject at a daily dose of about 5-80 mg, for example 5 mg, 10 mg, 15 mg, 20 mg, 25 mg, 30 mg, 35 mg, 40 mg, 45 mg, or 50 mg. The daily dose of the PLK1 inhibitor (e.g., onvansertib) can be adjusted (e.g., increased or decreased with the range) during the treatment (e.g., the first cycle, the second cycle, the third cycle, and a subsequent cycle) or during a single cycle (e.g., the first week, the second week and a subsequent week) of the treatment, for the subject. For example, the subject can be administered with 30 mg onvansertib on the first day or the first several days, with the option to decrease the dose to 25 mg, 20 mg, 15 mg, or 10 mg.

A maximum concentration (Cₘₐₓ) of the PLK1 inhibitor (e.g., onvansertib) in a blood of the subject (during the treatment or after the treatment) when the PLK1 inhibitor is administered alone or in combination with the anti-angiogenics can be from about 100 nmol/L to about 1500 nmol/L. For example, the Cₘₐₓ of the PLK1 inhibitor in a blood of the subject when the PLK1 inhibitor is administered alone or in combination with the anti-angiogenics can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the anti-angiogenics can be from about 1000 nmol/L·hour to about 400000 nmol/L·hour. For example, the AUC of a plot of a concentration of the PLK1 inhibitor in a blood of the subject over time (e.g., AUC₀₋₂₄ for the first 24 hours after administration) when the PLK1 inhibitor is administered alone or in combination with the anti-angiogenics can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of the PLK1 inhibitor (e.g., onvansertib) in a blood of the subject when the PLK1 inhibitor is administered alone or in combination with the anti-angiogenics can be from about 1 hour to about 5 hours. For example, the time (Tₘₐₓ) to reach a maximum concentration of the PLK1 inhibitor in a blood of the subject when the PLK1 inhibitor is administered alone or in combination with the anti-angiogenics can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with the anti-angiogenics can be from about 10 hours to about 60 hours. For example, the elimination half-life (T_{1/2}) of the PLK1 inhibitor in a blood of the subject when the PLK1 inhibitor is administered alone or in combination with the anti-angiogenics can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

As described herein, the methods, compositions and kits described herein can reduce or inhibit progression of the cancer. In some embodiments, the reduction or inhibition comprises inhibition of growth of one or more tumors in the subject and/or reducing the number of cancer cells detected in the subject by at least about 25%, 30%, 40%, 50%, 60%, or 70%; relative to an untreated subject. In some embodiments, the reduction or inhibition comprises inhibition of growth of one or more tumors in the subject and/or reducing the number of cancer cells detected in the subject by at least about 25%, 30%, 40%, 50%, 60%, or 70%; relative to patients who have received a prior bevacizumab treatment within about three months.

### Additional Cancer Therapeutics or Therapy

Methods, compositions and kits disclosed herein can be used for treating cancer. In some embodiments, a method for treating cancer (e.g., mCRC) comprises administrating an anti-angiogenics and a PLK1 inhibitor (e.g., onvansertib) to a subject (e.g., a patient) in need thereof, wherein the subjects have not received any treatment comprising bevacizumab within at least three months prior to the administration of the PLK1 inhibitor (e.g., onvansertib) and the anti-angiogenics (e.g., bevacizumab). In some embodiments, the patient has not received any treatment comprising bev within about 3 months prior to the administration of the PLK1 inhibitor (e.g., onvansertib) and the anti-angiogenics (e.g., bevacizumab). The patient has not been exposed to bevacizumab for at least or at least about 3 months.

The method can comprise administering a therapeutically effective amount of the anti-angiogenics and a therapeutically effective amount of the PLK1 inhibitor. The treatment can further comprise administration of at least one additional cancer therapeutics or cancer therapy.

Non-limiting examples of the additional cancer therapeutics or cancer therapy can include surgery, chemotherapy, radiation therapy (including external-beam, stereotactic, and intraoperative radiation therapy and brachytherapy), bone marrow transplant, immunotherapy, targeted drug therapy, cryoablation, or radiofrequency ablation. Where the cancer is colorectal cancer, examples of treatments include surgery, radiofrequency ablation, cryoablation, radiation therapy, chemotherapy (including medications comprising capecitabine, 5-fluorouracil (5-FU), irinotecan, oxaliplatin, trifluridine/tipiracil), targeted therapy (including anti-angiogenesis therapy using, for example bevacizumab, regorafenib, ziv-aflibercept, or ramucirumab; immunotherapy using, for example pembrolizumab, nivolumab, or ipilimumab; and PLK1 inhibitors).

The additional cancer therapeutics or cancer therapy can be a chemotherapy, for example, FOLFIRI, FOLFOX, XELOX (CAPOX), FOLFOXIRI, or a combination thereof. Chemotherapy regimens using fluorouracil are standard treatment for advanced colorectal cancer. Fluorouracil is a pyrimidine analog and antimetabolite, which incorporates into the DNA molecule and stops synthesis, thereby, preventing replication of cancer cells. Examples of these regimens include FOLFOX and FOLFIRI. The additional cancer therapeutics or cancer therapy can comprise a therapeutically effective amount of FOLFIRI. FOLFIRI is a chemotherapeutical cocktail, containing leucovorin (folinic acid), fluorouracil and irinotecan hydrochloride. Leucovorin is a vitamin B derivative and increases the cytotoxicity of fluorouracil in this combination. Irinotecan is a topoisomerase inhibitor, which prevents DNA from uncoiling and duplicating. FOLFIRI is often used in combination with other therapeutical reagents (e.g., bevacizumab) to improve efficacy and response rate.

FOLFOX is a chemotherapy regimen for treatment of colorectal cancer, made up of folinic acid, fluorouracil and oxaliplatin (Eloxatin). FOLFOX can be broken down into other subtypes such as FOLFOX-4, FOLFOX-6, and FOLFOX-7 depending on how these three drugs are administered. FOLFOX is usually used to treat colorectal cancer. It can also be used to treat pancreatic cancer and certain other cancers. FOLFOX is typically used as an adjuvant treatment (in addition to the primary therapy) for advanced cancers. However, FOLFOX can also be used as a first-line therapy for colorectal adenocarcinoma, which is the most common type of colon cancer. In this combination, oxaliplatin shows synergy with fluorouracil, with little toxicity overlap.

For decades, fluorouracil had been the only drug with demonstrated activity against colorectal cancer, commonly used in combination with leucovorin. Oxaliplatin and capecitabine are two relatively novel drugs used in the treatment of colorectal cancer. These drugs have been found to act synergistically, both in vivo and in vitro. The chemotherapy combination of oxaliplatin and capecitabine is known as XELOX, which is highly active in metastatic colorectal cancer (mCRC). Capecitabine has demonstrated high efficacy as first-line treatment for MCRC. It is an oral fluoropyrimidine that was rationally designed to generate FU preferentially at the tumor site, via a three-step enzymatic process that exploits the significantly higher activity of thymidine phosphorylase (TP) in tumors, compared with healthy tissue. Capecitabine causes less adverse effects, such as diarrhea, stomatitis, nausea, alopecia, and neutropenia, leading to less neutropenic fever/sepsis and associated hospitalizations compared to the combination therapy of leucovorin and fluorouracil (e.g., FOLFIRI, and FOLFOX), although hand-foot syndrome (HFS) occurred more frequently with capecitabine administration. Oxaliplatin is a third-generation cisplatin analog, and an organoplatinum complex, and is usually classified as alkylating agent although it is not capable of actually adding alkyl groups to DNA. Oxaliplatin is an integral component of the various fluorouracil regimens (e.g., FOLFOX), which have become a standard treatment for metastatic and node-positive colorectal cancer.

Another combination therapy of oxaliplatin and fluorouracil is known as FOLFOXIRI, containing leucovorin, fluorouracil, oxaliplatin and irinotecan. FOLFOXIRI is also an approved chemotherapy regimen for the treatment of advanced colorectal cancer and is often given with bevacizumab.

The additional cancer therapeutics can comprise FOLFIRI, bevacizumab, abiraterone, FOLFOX, an anti-EGFR agent, a KRAS directed inhibitor, gemcitabine, abraxane, nanoliposomal irinotecan, 5-FU, or a combination thereof. The PLK1 inhibitor and the cancer therapeutics or cancer therapy can be administered simultaneously or sequentially. In some embodiments, the additional cancer therapeutics or therapies comprises FOLFIRI, abiraterone, FOLFOX, an anti-EGFR agent, a KRAS directed inhibitor, gemcitabine, abraxane, nanoliposomal irinotecan, 5-FU, or a combination thereof. In some embodiments, anti-EGFR agents is optionally cetuximab. In some embodiments, the KRAS directed inhibitor is optionally a G12C inhibitor, a G12D inhibitor or a combination thereof. In some embodiments, the additional cancer therapy is FOLFOX (leucovorin, fluorouracil, and oxaliplatin), FOLFIRI (leucovorin, fluorouracil, and irinotecan), or a combination thereof.

### Methods for Predicting/Determining Treatment Efficacy and Status for Cancer

Disclosed herein include methods of treating cancer. In some embodiments, the method comprises: administering a PLK1 inhibitor and an anti-angiogenics to a subject with a metastatic cancer, thereby reducing or inhibiting progression of the metastatic cancer wherein the subject or the subjects have not received any treatment comprising bevacizumab within at least three months prior to the administration of the PLK1 inhibitor (e.g., onvansertib) and the anti-angiogenics (e.g., bevacizumab). In some embodiments, the patient has not received any treatment comprising bevacizumab within about 3 months prior to the administration of the PLK1 inhibitor (e.g., onvansertib) and the anti-angiogenics (e.g., bevacizumab). The patient has not been exposed to bevacizumab for at least about 3 months. The method described herein using the combination of the anti-angiogenics and the PLK1 inhibitor is expected to be effective with various metastatic cancers, for example metastatic colorectal cancer, metastatic bladder cancer, metastatic breast cancer, metastatic kidney cancer, metastatic lung cancer, metastatic ovarian cancer, metastatic pancreatic cancer, metastatic prostate cancer, metastatic stomach cancer, metastatic thyroid cancer, metastatic uterine cancer, metastatic renal cancer, metastatic cervical cancer, metastatic recurrent glioblastoma, or a combination thereof.

The method can comprise one or more of (1) determining cancer status of the subject, (2) determining responsiveness of the subject to a PLK1 inhibitor treatment, and (3) administering one or more cancer therapeutics or therapies for the cancer.

In some embodiments, administering the PLK1 inhibitor and the anti-angiogenics improves one or more therapeutic effects in the treated subjects relative to a control or a baseline. The one or more therapeutic effects can comprise size of a tumor derived from the metastatic cancer, objective response rate (ORR), duration of response, time to response, progression free survival (PFS), overall survival (OS), disease control rate (DCR), oncogenic allelic burden, or a combination thereof. In some embodiments, administering the PLK1 inhibitor and the anti-angiogenics improves the ORR in the subjects, improves PFS in the treated subjects, improves OS in the treated subjects, improves DCR in the treated subjects, reduces oncogenic allelic burden in the treated subjects, or a combination thereof, relative to subjects who have received prior treatment comprising inhibiting angiogenesis.

Disclosed herein include methods of improving objective response rate (ORR), progression free survival (PFS), or both in subjects with a metastatic cancer. In some embodiments, the method comprises administering a PLK1 inhibitor and an anti-angiogenics to the subjects, thereby improving the ORR and/or the PFS of the subjects, wherein the subject or the subjects have not received any treatment comprising bevacizumab within at least three months prior to the administration of the PLK1 inhibitor (e.g., onvansertib) and the anti-angiogenics (e.g., bevacizumab). For example, the subject has not been exposed to bevacizumab for at least three months (e.g., 3 months, 4 months, 5 months, 6 months, 7 months, 8 months, 9 months, 10 months, 11 months, 1 year, 2 years, 3 years or longer).

In some embodiments, administering the PLK1 inhibitor and the anti-angiogenics synergistically improves the ORR and/or the PFS of the treated subjects relative to the PLK1 inhibitor treatment alone, the anti-angiogenics treatment alone, and/or the additive effect of the PLK1 inhibitor treatment alone and the anti-angiogenics treatment alone.

Tumor evaluations and assessment of tumor burden and therapeutic efficacy can be made based on RECIST criteria (Therasse et al 2000), New Guidelines to Evaluate the Response to Treatment in Solid Tumors, Journal of National Melanoma Institute, Vol. 92; 205-16 and is made within the presently disclosed methods, in some embodiments, according to the revised RECIST guidelines (version 1.1) (Eisenhauer et al 2009, New response evaluation criteria in solid tumors: revised RECIST guideline (version 1.1). Eur J Melanoma, 45(2):228-47.), which is hereby incorporated by reference in its entirety.

In some embodiments, the treatment results in a sustained response in the subject after cessation of the treatment. “Sustained response” can refer to the sustained effect on reducing tumor growth after cessation of a treatment. For example, the tumor size may remain the same or smaller as compared to the size at the beginning of the administration phase. In some embodiments, the sustained response has a duration at least the same as the treatment duration, at least 1.5×, 2.0×, 2.5×, or 3.0×length of the treatment duration.

The treatment methods disclosed herein may result in a partial or complete response. As used herein, “complete response” or “CR” can refer to disappearance of all target lesions; “partial response” or “PR” can refer to at least a 30% decrease in the sum of the longest diameters (SLD) of target lesions, taking as reference the baseline SLD; and “stable disease” or “SD” can refer to neither sufficient shrinkage of target lesions to qualify for PR, nor sufficient increase to qualify for PD, taking as reference the smallest SLD since the treatment started. As used herein, “objective response rate” (ORR) can refer to the sum of complete response (CR) rate and partial response (PR) rate.

The treatment methods disclosed herein can lead to an increase in progression free survival (PFS) and overall survival (OS) of the subjects administered the combination therapy. As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease. As used herein, “overall survival” refers to the percentage of subjects in a group who are likely to be alive after a particular duration of time. As used herein, “disease control rate” refers to complete response (CR) plus partial response (PR) plus stable disease (SD).

In some embodiments, administering the PLK1 inhibitor and the anti-angiogenics improves the ORR in the subjects by, by about, by at least, or by at least about 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) relative to subjects who have received prior treatment comprising inhibiting angiogenesis (e.g., bevacizumab) within about three months prior to the administration of the PLK1 inhibitor and the anti-angiogenics. In some embodiments, administering the PLK1 inhibitor and the anti-angiogenics improves the ORR in the subjects by, by about, by at least, or by at least about 2-fold, 3-fold, 4-fold, 5-fold or higher relative to subjects who have received prior treatment comprising anti-angiogenesis (e.g., bevacizumab) within about three months (e.g., within 3 months, within 2 months, within 1 month) prior to the administration of the PLK1 inhibitor and the anti-angiogenics. In some embodiments, administering the PLK1 inhibitor and the anti-angiogenics improves the PFS in the subjects by, by about, by at least, or by at least about 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) relative to subjects who have received prior treatment comprising inhibiting angiogenesis (e.g., bevacizumab) within about three months prior to the administration of the PLK1 inhibitor and the anti-angiogenics. In some embodiments, administering the PLK1 inhibitor and the anti-angiogenics improves the PFS in the subjects by, by about, by at least, or by at least about 2-fold, 3-fold, 4-fold, 5-fold or higher relative to subjects who have received prior treatment comprising inhibiting angiogenesis (e.g., bevacizumab) within about three months (e.g., within 3 months, within 2 months, within 1 month) prior to the administration of the PLK1 inhibitor and the anti-angiogenics.

In some embodiments, administering a PLK1 inhibitor and an anti-angiogenics to a subject wherein the subject has not received any treatment comprising bevacizumab within at least three months prior to the administration of the PLK1 inhibitor (e.g., onvansertib) and the anti-angiogenics (e.g., bevacizumab) can result in an ORR of, of at least, of about, or of at least about 33% (e.g., 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values). In some embodiments, administering a PLK1 inhibitor and an anti-angiogenics to a subject who has received any prior treatment comprising inhibiting angiogenesis can result in an ORR of about 10% or less (e.g., 1%, 2%, 3%, 4%, 5%, 6%, 7%, 8%, 9%, 10%, or a number or a range between any two of these values). In some embodiments, the patient has not received any treatment comprising bevacizumab within about 3 months prior to the administration of the PLK1 inhibitor (e.g., onvansertib) and the anti-angiogenics (e.g., bevacizumab). The patient has not been exposed to bevacizumab for at least or at least about 3 months.

In some embodiments, administering a PLK1 inhibitor and an anti-angiogenics to a subject wherein the subject has not received any treatment comprising bevacizumab within about three months prior to the administration of the PLK1 inhibitor (e.g., onvansertib) and the anti-angiogenics (e.g., bevacizumab) can result in an median PFS of, of at least, of about, or of at least about 10 months (e.g., 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years). In some embodiments, administering a PLK1 inhibitor and an anti-angiogenics to a subject wherein the subject has not received any treatment comprising bevacizumab within about three months prior to the administration of the PLK1 inhibitor (e.g., onvansertib) and the anti-angiogenics (e.g., bevacizumab) can result in a median PFS of about 6 months or less (e.g., 1 month, 2 months, 3 months, 4 months, 5 months, 6 months, or a number or a range between any two of these values).

The term “inhibition of tumor growth” or “reduced tumor growth” can refer to causing a reduction in or complete cessation of tumor growth and/or causing a regression in tumor size (e.g., diameter and/or volume). The term “tumor volume” or “tumor size” can refer to the total size of the tumor, which can include the tumor itself plus affected lymph nodes if applicable. The presence or absence of a tumor, and tumor size can be determined by a variety of methods known in the art, such as, e.g. by measuring the dimensions of the tumor using calipers, computed tomography (CT) or magnetic resonance imaging (MRI) scans, mammography, and X-ray. The volume can be calculated using equations based on, for example, the z-axis diameter, or on standard shapes such as the sphere, ellipsoid, or cube. Tumor size may be assessed at any time before, during or following at least one cycle of treatment with onvansertib and/or anti-angiogenics. Tumor size can be assessed at a first time point, and at one or more additional time points. In some embodiments, tumor size can be assessed in the subject and, e.g., an untreated subject at equivalent time points (e.g., at a first time point, and at one or more additional time points). Tumor growth can be determined by, e.g., measuring tumor size at a first time point and measuring tumor size at one or more additional time points. In some embodiments, increased inhibition of tumor growth in the subject (e.g., improved ORR) indicates the subject as responsive to the cancer treatment.

The inhibition of growth of at least one of one or more tumors in the subject can be, can be about, can be at least, or can be at least about 1.1 times greater, 1.2 times greater, 1.3 times greater, 1.4 times greater, 1.5 times greater, 1.6 times greater, 1.7 times greater, 1.8 times greater, 1.9 times greater, 2 times greater, or a number or a range between any two of these values, or more, than the inhibition of growth caused by onvansertib and the anti-angiogenics in a subject who has received previous treatment for inhibiting angiogenesis within about 3 months prior to the administration of onvansertib and the anti-angiogenics, following one or more cycles of treatment. The inhibition of growth of at least one of the one or more tumors in the subject can be increased by, by about, by at least, or by at least about 25%, 30%, 35%, 40%, 50%, 55%, 60%, 65%, 70%, 75%, 80%, 85%, 90%, 95%, 100% or a number or a range between any two of these values, relative to a subject who has received previous treatment for inhibiting angiogenesis within about 3 months prior to the administration of onvansertib and the anti-angiogenics, following one or more cycles of treatment. The growth of at least one of the one or more tumors in the subject can be inhibited by, by about, by at least, or by at least about 50%, 55%, 60%, 65%, 70%, 75%, 80%, 85%, 90%, 95%, 100% or a number or a range between any two of these values relative to an untreated subject or a subject who has received prior anti-angiogenic treatment within about 3 months prior to the administration of onvansertib and the anti-angiogenics, following one or more cycles of treatment with the combination. The growth of at least one of the one or more tumors in the subject can be inhibited by, by about, by at least, or by at least about 70%, 75%, 80%, 85%, 90%, 95%, 100% or a number or a range between any two of these values relative to an untreated subject, following one or more cycles of treatment. The subject can be tumor-free following one or more cycles of treatment.

The inhibition of growth of at least one of the one or more tumors in the subject can be, can be about, can be at least, or can be at least about 1.1 times greater, 1.2 times greater, 1.3 times greater, 1.4 times greater, 1.5 times greater, 1.6 times greater, 1.7 times greater, 1.8 times greater, 1.9 times greater, 2 times greater, or a number or a range between any two of these values, or more, than the inhibition of growth caused by onvansertib alone or the anti-angiogenics alone, following one or more cycles of treatment. The inhibition of growth of at least one of the one or more tumors in the subject can be increased by, by about, by at least, or by at least about 25%, 30%, 35%, 40%, 50%, 55%, 60%, 65%, 70%, 75%, 80%, 85%, 90%, 95%, 100% or a number or a range between any two of these values, relative to a subject treated with onvansertib alone or the anti-angiogenics alone, following one or more cycles of treatment. The growth of at least one of the one or more tumors in the subject can be inhibited by, by about, by at least, or by at least about 50%, 55%, 60%, 65%, 70%, 75%, 80%, 85%, 90%, 95%, 100% or a number or a range between any two of these values relative to an untreated subject, following one or more cycles of treatment. The growth of at least one of the one or more tumors in the subject can be inhibited by, by about, by at least, or by at least about 70%, 75%, 80%, 85%, 90%, 95%, 100% or a number or a range between any two of these values relative to an untreated subject, following one or more cycles of treatment. The subject can be tumor-free following one or more cycles of treatment.

In some embodiments, the first time point is prior or immediately prior to the combination treatment, and at least one of the one or more additional time points are at the end of or after at least one cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment.

In some embodiments, the first cycle of the combination treatment is immediately prior to the second cycle of the combination treatment. In some embodiments, the method comprises continuing the combination treatment to the subject if the subject is indicated as responsive to the combination treatment. In some embodiments, the method comprises discontinuing the combination treatment to the subject and/or starting a different combination treatment to the subject if the subject is not indicated as responsive to the combination treatment.

The first time point can be prior or immediately prior to the combination treatment, and the one or more additional time points are at the end of or after at least a cycle of the combination treatment, optionally the cycle of the combination treatment is the first cycle of the combination treatment. In some embodiments, the first time point is prior or immediately prior to a first cycle of the combination treatment, and the one or more additional time points are at the end of or after a second cycle of the combination treatment, optionally the first cycle of the combination treatment is immediately prior to the second cycle of the combination treatment.

Determining the responsiveness of the subject can comprise determining if the subject is a responder of the treatment, if the subject is or is going to be in complete recovery (CR), or if the subject is or is going to be in partial remission (PR). Determining the responsiveness of the subject can comprise determining objective response rate (ORR), duration of response, time to response, progression free survival (PFS), overall survival (OS), disease control rate (DCR), oncogenic allelic burden, or a combination thereof of the subject. Determining the responsiveness of the subject can comprise determining if the subject has a partial response to the treatment, if the subject has a complete response to the treatment, if the subject has a stable disease (SD) status, or if the subject has a progressive disease (PD) status. In some embodiments, the method comprises starting an additional treatment to the subject if the subject is indicated as in cancer relapse. The additional treatment can be the same or different from the current or prior combination treatment.

Additional methods for assessing cancer status of the subject include determining ECOG status. As used herein, ECOG status refers to Eastern Cooperative Oncology Group (ECOG) Performance Status (Oken M, et al Toxicity and response criteria of the Eastern Cooperative Oncology Group. Am J Clin Oncol 1982; 5(6):649-655), as shown below: 0, Fully active, able to carry on all pre-disease performance without restriction; 1, Restricted in physically strenuous activity but ambulatory and able to carry out work of a light or sedentary nature, e.g., light house work, office work; 2, Ambulatory and capable of all selfcare but unable to carry out any work activities; up and about more than 50% of waking hours; 3, Capable of only limited selfcare; confined to bed or chair more than 50% of waking hours; 4, Completely disabled; cannot carry on any selfcare; totally confined to bed or chair; and 5, Dead.

As described herein, the patient can achieve complete response or partial response after treatment with the anti-angiogenics and the PLK1 inhibitor. In some embodiments, the patient achieves a complete response. In some embodiments, the patient achieves a partial response.

Disclosed herein include methods, compositions, kits, and systems for predicting/determining clinical outcome for a combination treatment of cancer of the present disclosure, monitoring of the combination treatment, predicting/determining responsiveness of a subject to the combination treatment, determining the status of the cancer in a subject, wherein the subjects have not received any treatment comprising bevacizumab within at least three months prior to the administration of the PLK1 inhibitor (e.g., onvansertib) and the anti-angiogenics (e.g., bevacizumab), and improving combination treatment outcome. In some embodiments, the patient has not received any treatment comprising bevacizumab within about 3 months prior to the administration of the PLK1 inhibitor (e.g., onvansertib) and the anti-angiogenics (e.g., bevacizumab). The patient has not been exposed to bevacizumab for at least about 3 months. The methods, compositions, kits and systems can be used to guide the combination treatment, provide combination treatment recommendations, reduce or avoid unnecessary ineffective combination treatment for patients. In some embodiments, the combination treatment disclosed herein can improve oncogenic allelic burden in a subject. As used herein, “allelic burden” can refer to the ratio between mutant (e.g., oncogenic) and wild-type alleles in clinical samples employed for genotyping. In some embodiments, the sample can comprise circulating tumor DNA (ctDNA). ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment. Such analysis of ctDNA has been described in WO2021146322, the content of which is incorporated herein by reference in its entirety.

A method of determining responsiveness of a subject to a combination treatment comprising an anti-angiogenics and a PLK1 inhibitor of the disclosure can comprise, for example, analyzing ctDNA of a subject with cancer, wherein the subject is undergoing a treatment and/or has received the combination treatment and has not received any treatment comprising bevacizumab within at least three months prior to the administration of the PLK1 inhibitor (e.g., onvansertib) and the anti-angiogenics (e.g., bevacizumab), thereby determining the responsiveness of the subject to the combination treatment. In some embodiments, the patient has not received any treatment comprising bevacizumab within about 3 months prior to the administration of the PLK1 inhibitor (e.g., onvansertib) and the anti-angiogenics (e.g., bevacizumab). In some embodiments, determining the responsiveness of the subject comprises determining if the subject is a responder of the treatment, if the subject is or is going to be in CR, or if the subject is or is going to be in partial remission (PR). For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the cancer treatment.

In some embodiments, the first time point is prior or immediately prior to the combination treatment, and at least one of the one or more additional time points are at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment.

In some embodiments, the first cycle of the combination treatment is immediately prior to the second cycle of the combination treatment. In some embodiments, the method comprises continuing the combination treatment to the subject if the subject is indicated as responsive to the combination treatment. In some embodiments, the method comprises discontinuing the combination treatment to the subject and/or starting a different combination treatment to the subject if the subject is not indicated as responsive to the combination treatment.

Disclosed herein include methods of determining cancer status of a subject, wherein the subjects have not received any treatment comprising bevacizumab within at least three months prior to the administration of the PLK1 inhibitor (e.g., onvansertib) and the anti-angiogenics (e.g., bevacizumab), comprising analyzing ctDNA of a subject, thereby determining cancer status of the subject. The subject can be a subject undergoing a current combination treatment comprising an anti-angiogenics and a PLK1 inhibitor of the present disclosure, a subject that has received a prior combination treatment of the present disclosure, and/or a subject that is in remission for the cancer. The subject in remission for cancer can be in complete remission (CR), or in partial remission (PR).

In some embodiments, analyzing the ctDNA comprises detecting variant allele frequency in the ctDNA. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, an increase in the variant allele frequency at the additional sample(s) relative to the first sample indicates that the subject is at risk of cancer relapse or is in cancer relapse.

In some embodiments, the first time point is prior or immediately prior to the combination treatment, and the one or more additional time points are at the end of or after at least a cycle of the combination treatment, optionally the cycle of the combination treatment is the first cycle of the combination treatment. In some embodiments, the first time point is prior or immediately prior to a first cycle of the combination treatment, and the one or more additional time points are at the end of or after a second cycle of the combination treatment, optionally the first cycle of the combination treatment is immediately prior to the second cycle of the combination treatment.

In some embodiments, the method comprises starting an additional treatment to the subject if the subject is indicated as in cancer relapse. The additional treatment can be the same or different from the current or prior combination treatment.

The variant allele frequency in ctDNA can be determined, for example, by total mutation count in the ctDNA in each of the first sample and one or more additional samples, or by the mean variant allele frequency in each of the first sample and one or more additional samples. In some embodiments, the variant allele frequency is mutant allelic frequency (MAF) for a driver mutation of the cancer (e.g., metastatic colorectal cancer, metastatic bladder cancer, metastatic breast cancer, metastatic kidney cancer, metastatic lung cancer, metastatic ovarian cancer, metastatic pancreatic cancer, metastatic prostate cancer, metastatic stomach cancer, metastatic thyroid cancer, metastatic uterine cancer, metastatic renal cancer, metastatic cervical cancer, metastatic recurrent glioblastoma, or a combination thereof). In some embodiments, the variant allele frequency is MAF for one or more driver mutations of the cancer (e.g., metastatic colorectal cancer, metastatic bladder cancer, metastatic breast cancer, metastatic kidney cancer, metastatic lung cancer, metastatic ovarian cancer, metastatic pancreatic cancer, metastatic prostate cancer, metastatic stomach cancer, metastatic thyroid cancer, metastatic uterine cancer, metastatic renal cancer, metastatic cervical cancer, metastatic recurrent glioblastoma, or a combination thereof). In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is, or is about, 0.01 to −0.10. In some embodiments, the MAF threshold is, or is about, 0.06. In some embodiments, the MAF threshold is, or is about, 0.05.

In some embodiments, the first sample comprises ctDNA from the subject before treatment, and the one of additional samples comprises ctDNA from the subject after treatment.

The driver mutation can be, or can comprise, a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PH1F6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2. In some embodiments, at least one of the one or more the driver mutations is a mutation in in the 75 genes. In some embodiments, one or more the driver mutations are mutations in the 75 genes. The driver mutation or at least one of the one or more driver mutations can be in a gene selected from TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the method further comprises determining variant allele frequency in one or more of the ctDNA, PBMCs and BMMCs of the subject.

The ctDNA can be analyzed using, for example, polymerase chain reaction (PCR), next generation sequencing (NGS), and/or droplet digital PCR (ddPCR). The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include methods of improving treatment outcome for the cancer. The method can comprise: detecting variant allele frequency in ctDNA obtained from a subject at a first time point in a first sample before the subject undergoes a combination treatment comprising an anti-angiogenics and a PLK1 inhibitor of the present disclosure; detecting variant allele frequency in ctDNA obtained from the subject at one or more additional time points in one or more additional samples after the subject undergoes the combination treatment; determining the difference of the variant allele frequency in ctDNA between the first and at least one of the one or more additional samples, a decrease in the oncogenic allelic burden and/or variant allele frequency in at least one of the additional samples relative to the first sample indicates the subject as responsive to the combination treatment; and continuing the combination treatment to the subject if the subject is indicated as responsive to the combination treatment, or discontinuing the combination treatment to the subject and/or starting a different cancer treatment to the subject if the subject is not indicated as responsive to the combination treatment.

Also disclosed herein include methods of treating cancer. The method can comprise: administering a combination treatment comprising an anti-angiogenics and a PLK1 inhibitor of the present disclosure to a subject in need thereof; determining a decrease, relative to an oncogenic allelic burden and/or variant allele frequency in a first sample of the subject obtained at a first time point before the subject receives the combination treatment, in a variant allele frequency in a second sample of the subject obtained at a second time point after the subject receives the combination treatment; and continuing with the combination treatment. In some embodiments, the subject is a subject newly diagnosed with cancer, for example a subject that has not received any treatment comprising bevacizumab within at least three months prior to the administration of the PLK1 inhibitor (e.g., onvansertib) and the anti-angiogenics (e.g., bevacizumab). In some embodiments, the subject has not received any treatment comprising bevacizumab within at least three months prior to the administration of the PLK1 inhibitor (e.g., onvansertib) and the anti-angiogenics (e.g., bevacizumab) and was in remission for the cancer, for example a subject in complete remission (CR), or in partial remission (PR) after receiving the prior combination treatment. In some embodiments, the prior treatment does not comprise the use of an anti-angiogenics, a PLK1 inhibitor, or both.

The first time point can be, for example, prior or immediately prior to the combination treatment. The at least one of the one or more additional time points can be, for example, at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment. In some embodiments, the first cycle of the combination treatment is immediately prior to the second cycle of the combination treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of the cancer (e.g., metastatic colorectal cancer, metastatic bladder cancer, metastatic breast cancer, metastatic kidney cancer, metastatic lung cancer, metastatic ovarian cancer, metastatic pancreatic cancer, metastatic prostate cancer, metastatic stomach cancer, metastatic thyroid cancer, metastatic uterine cancer, metastatic renal cancer, metastatic cervical cancer, metastatic recurrent glioblastoma, or a combination thereof). In some embodiments, the variant allele frequency is MAF for one or more driver mutations of the cancer (e.g., metastatic colorectal cancer, metastatic bladder cancer, metastatic breast cancer, metastatic kidney cancer, metastatic lung cancer, metastatic ovarian cancer, metastatic pancreatic cancer, metastatic prostate cancer, metastatic stomach cancer, metastatic thyroid cancer, metastatic uterine cancer, metastatic renal cancer, metastatic cervical cancer, metastatic recurrent glioblastoma, or a combination thereof). In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is −0.05.

The method can further comprise determining variant allele frequency in one or more of the ctDNA, PBMCs and BMMCs of the subject. The variant allele frequency in ctDNA can be detected, for example, using polymerase chain reaction (PCR) or next generation sequencing (NGS). In some embodiments, the variant allele frequency in ctDNA is detected using droplet digital PCR (ddPCR). At least one of the first sample, the one or more additional samples, and the second sample can be derived from whole blood of the subject, plasma of the subject, serum of the subject, or a combination thereof. In some embodiments, the ctDNA is from whole blood of the subject, plasma of the subject, serum of the subject, or a combination thereof.

In some embodiments, the subject whose ctDNA is analyzed is undergoing or will be undergoing treatment for the cancer. The method can comprise analyzing ctDNA of the subject before the treatment. The treatment can comprise one or more cycles, and the ctDNA is analyzed before, during and after one or more cycles of the treatment. For example, the ctDNA can be analyzed before, during and after two or more cycle of the treatment, three or more cycle of the treatment, or each cycle of the treatment. Each cycle of treatment can be at least 21 days, for example, 21 days, 22 days, 23 days, 24 days, 25 days, 26 days, 27 days, 28 days, 29 days, 30 days, or more, or a range between any two of these values. In some embodiments, each cycle of treatment is from about 21 days to about 28 days. In some embodiments, each cycle of treatment is from 21 days to 28 days. In some embodiments, the subject is human.

### Compositions and Kits

Disclosed herein include compositions and kits for treating cancer. In some embodiments, the kit comprises: a Polo-like kinase 1 (PLK1) inhibitor; and a manual providing instructions for administrating the PLK1 inhibitor with an anti-angiogenics to a subject for treating a metastatic cancer, wherein the subject has not received any treatment comprising bevacizumab within at least three months prior to the administration of the PLK1 inhibitor (e.g., onvansertib) and the anti-angiogenics (e.g., bevacizumab). In some embodiments, the patient has not received any treatment comprising bevacizumab within about 3 months prior to the administration of the PLK1 inhibitor (e.g., onvansertib) and the anti-angiogenics (e.g., bevacizumab). The patient has not been exposed to bevacizumab for at least about 3 months. In some embodiments, the kit comprises the anti-angiogenics. For example, the patient has not been exposed to bev for at least 3 months. The cancer can be, for example, metastatic colorectal cancer, metastatic bladder cancer, metastatic breast cancer, metastatic kidney cancer, metastatic lung cancer, metastatic ovarian cancer, metastatic pancreatic cancer, metastatic prostate cancer, metastatic stomach cancer, metastatic thyroid cancer, metastatic uterine cancer, metastatic renal cancer, metastatic cervical cancer, metastatic recurrent glioblastoma or a combination thereof. In some embodiments, the PLK1 inhibitor is onvansertib and the anti-angiogenics is bevacizumab.

In some embodiments, the instructions comprise instructions for administrating the PLK1 inhibitor and the anti-angiogenics simultaneously. In some embodiments, the instructions comprise instructions for administrating the PLK1 inhibitor and the anti-angiogenics sequentially. In some embodiments, the instructions comprise instructions for administering of the PLK1 inhibitor orally. In some embodiments, the instructions comprise instructions for administrating the anti-angiogenics orally. In some embodiments, the instructions comprise instructions for administrating the anti-angiogenics intravenously.

The instructions can comprise instructions wherein a subject is known to have received a treatment comprising bevacizumab at least three months prior to the administration of the PLK1 inhibitor (e.g., onvansertib) and the anti-angiogenics (e.g., bevacizumab). In some embodiments, the patient has received a treatment comprising bev at least 3 months, 4 months, 5 months, 6 months, 7 months, 8 months, 9 months, 10 months, 11 months, 1 year, 2 years, 3 years, 4 year, 5 years, 6 years, 7 years, 8 years, 9 years, 10 years or a number or a range between any two of these values, prior to the administration of the PLK1 inhibitor (e.g., onvansertib) and the anti-angiogenics (e.g., bevacizumab).

The instructions can comprise instructions the subject has not received any treatment comprising bevacizumab within at least three months prior to the administration of the PLK1 inhibitor (e.g., onvansertib) and the anti-angiogenics (e.g., bevacizumab). In some embodiments, the patient has not received any treatment comprising bevacizumab within about 3 months prior to the administration of the PLK1 inhibitor (e.g., onvansertib) and the anti-angiogenics (e.g., bevacizumab). In some embodiments, the prior treatment does not comprise the use of an anti-angiogenics, a PLK1 inhibitor, or both. In some embodiments, the instructions comprise instructions the subject was in remission for the cancer. In some embodiments, the subject in remission for cancer was in complete remission (CR), or in partial remission (PR).

In some embodiments, the instructions comprise instructions for administering each of the anti-angiogenics and the PLK1 inhibitor to the subject in a cycle of at least twice within a week. In some embodiments, the instructions comprise instructions for administering each of the anti-angiogenics and the PLK1 inhibitor to the subject in a cycle of at least five times within a week In some embodiments, the instructions comprise instructions for administering the anti-angiogenics, the PLK1 inhibitor, or both are in a cycle of at least 7 days. In some embodiments, each cycle of treatment is at least about 21 days. In some embodiments, each cycle of treatment is from about 21 days to about 28 days, for example 28 days. In some embodiments, the instructions comprise instructions for administering the PLK1 inhibitor on at least four days in the cycle. In some embodiments, the instructions comprise instructions for not administering the PLK1 inhibitor on at least one day in the cycle. In some embodiments, the instructions comprise instructions for administrating the anti-angiogenics daily. In some embodiments, the instructions comprise instructions for administrating the anti-angiogenics and the PLK1 inhibitor for at least two cycles. The instructions can comprise instructions for administrating the anti-angiogenics daily, weekly, bi-weekly, every three weeks, every four weeks, or monthly.

The anti-angiogenics can be capable of inhibiting VEGF-A, VEGFR-1, VEGFR-2, VEGFR-3, EGFR, HER2, PDGFR family proteins, RAF, Kit (or c-Kit), FLT3, CSF-1R, RET, Abl, Itk, LcK, c-FMS, FGFR family proteins, c-Met, PlGF, TNF-α, IFNs, ILs, bFGF, mTOR, or any combination thereof. The anti-angiogenics (e.g., angiogenic inhibitor) can be Afatinib (Gilotrif©), Axitinib (Inlyta®), Bevacizumab (Avastin®), Cabozantinib (Cometriq®), Cetuximab (Erbitux®), Erlotinib (Tarceva®), Everolimus (Afinitor®), Gefitinib (Iressa®), Imatinib (Gleevec®), Lapatinib (Tykerb®), Lenalidomide (Revlimid®), Lenvatinib mesylate (Lenvima®), Necitumumab (Portrazza™), Neratinib (Nerlynx®), Panitumumab (Vectibix®), Pazopanib (Votrient®), Pertuzumab (Perjeta®), Ramucirumab (Cyramza®), Regorafenib (Stivarga®), Sorafenib (Nexavar®), Sunitinib (Sutent®), Thalidomide (Synovir, Thalomid®), Trastuzumab (Ontruzant®), Vandetanib (Caprelsa®), or Ziv-aflibercept (Zaltrap®).

The PLK1 inhibitor can be selective and/or specific for PLK1. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI6727), GSK461364, AZD1775, CYC140, HMN-176, HMN-214, rigosertib (ON-01910), MLN0905, TKM-080301, TAK-960 or Ro3280. In some embodiments, the PLK1 inhibitor is onvansertib. In some embodiments, the anti-angiogenics is bevacizumab, and the PLK1 inhibitor is onvansertib. In some embodiments, the instructions comprise instructions for administering the PLK1 inhibitor at 12 mg/m²-90 mg/m², or 5-60 mg daily. In some embodiments, the instructions comprise instructions for administering the anti-angiogenics at 20 mg-1200 mg.

### EXAMPLES

Some aspects of the embodiments discussed above are disclosed in further detail in the following examples, which are not in any way intended to limit the scope of the present disclosure.

### Example 1

### Patients and General Methods

This example briefly describes guidelines for patient selection and general methods used in the clinical trial (NCT03829410). Additional information about the methods and patients is available in Ahn et al. J Clin Oncol. 2024 Oct. 30:JC02401266. doi: 10.1200 JCO-24-01266, the content of which is incorporated herein by reference in its entirety.

### Study Design and Patients

The study presented in the clinical trial was an open-label, single-arm phase Ib/II study conducted at seven sites in the United States, evaluating the efficacy and tolerability of onvansertib in combination with FOLFIRI and bevacizumab in second-line treatment of KRAS-mutant mCRC. The phase I portion, previously reported, described detailed eligibility criteria. Briefly, patients age ≥18 years with histologically confirmed metastatic and unresectable CRC and a KRAS mutation in exons 2, 3, or 4 detected in the primary tumor or the metastasis by a Clinical Laboratory Improvement Amendments-certified laboratory were eligible. Patients were required to have received ≥6 weeks of oxaliplatin and fluoropyrimidine, with or without bevacizumab, and to have either progressed within 6 months of treatment or shown intolerance to oxaliplatin. Eastern Cooperative Oncology Group performance status of 0 or 1 and adequate organ function were required. Key exclusion criteria included microsatellite instability-high/mismatch repair deficiency, BRAF V600 mutation, more than one prior chemotherapy regimen for metastatic disease, and untreated brain metastasis.

The study protocol was approved by each participating site's institutional review board and/or independent ethics committee and conducted in accordance with the Declaration of Helsinki and Good Clinical Practice guidelines. All participants provided written informed consent.

### Treatment

Onvansertib was administrated orally at the recommended phase 2 dose (RP2D) of 15 mg/m² once daily on days 1-5 and 15-19 of a 28-day cycle in combination with FOLFIRI (irinotecan 180 mg/m² intravenous (IV); leucovorin 400 mg/m² IV; fluorouracil (FU) 400 mg/m² IV bolus; FU 2,400 mg/m² continuous IV infusion for 46 hours) and bevacizumab (5 mg/kg IV) administered once daily on days 1 and 15 until disease progression, death, unacceptable toxicity, or withdrawal from the study. The elimination of the FU bolus and leucovorin was allowed at the investigator's discretion.

### End Points

The primary end point of the phase II study which was the investigator-assessed ORR according to RECIST version 1.1 (v1.1) in patients who received at least one cycle (28 days) of treatment. Secondary efficacy end points included disease control rate (percentage of patients with best overall response being complete response [CR], partial response [PR] or stable disease [SD]), PFS (time from treatment initiation to progression or death, whichever occurs first), duration of response (DOR, time from first response [CR or PR] to progression or death, whichever occurs first), and reduction in KRAS-mutant allelic burden in liquid biopsies.

### Assessment

Tumor response was assessed using computed tomography or magnetic resonance imaging per RECIST v.1.1, at screening and during treatment at every other cycle until end of treatment. For patients who discontinued study treatment for reasons other than progressive disease, follow-up information was collected until the patient started new therapy, underwent a procedure, experienced progressive disease, or died, for up to 1 year.

Safety assessments in patients who received at least one treatment dose included physical examinations, laboratory test results, electrocardiograms, and monitoring of adverse events (AEs) graded by the National Cancer Institute Common Terminology Criteria for

### Adverse Events Version 5.0.

Blood samples were collected at baseline and after completion of cycle 1 to analyze KRAS-mutant circulating tumor DNA (ctDNA) as described previously.15 For patients with available archival tumor tissue, the presence of KRAS mutations was tested using the Tempus xT assay (Tempus Labs Inc, Chicago, IL).

### Example 2

### Time Effect on the Clinical Efficacy of Onvansertib+FOLFIRI+Bevacizumab

This example assesses the association between time since bevacizumab first-line treatment and efficacy of the combination of onvansertib, FOLFIRI and bevacizumab in the bev-exposed patients of the clinical trial study NCT03829410 described in Example 1.

Analysis of the clinical data previously showed that bevacizumab-exposed patients are less sensitive to onvansertib combined with FOLFIRI+bevacizumab compared to bevacizumab-naïve patients (FIG. 2), suggesting that bevacizumab may confer resistance to the combination. The translational research supports that bevacizumab exposure may result in transcriptional changes that contribute to resistance to both onvansertib and bevacizumab. It is speculated that the resistance associated with bevacizumab treatment may be reverted in tumors not exposed to bevacizumab for a period of time.

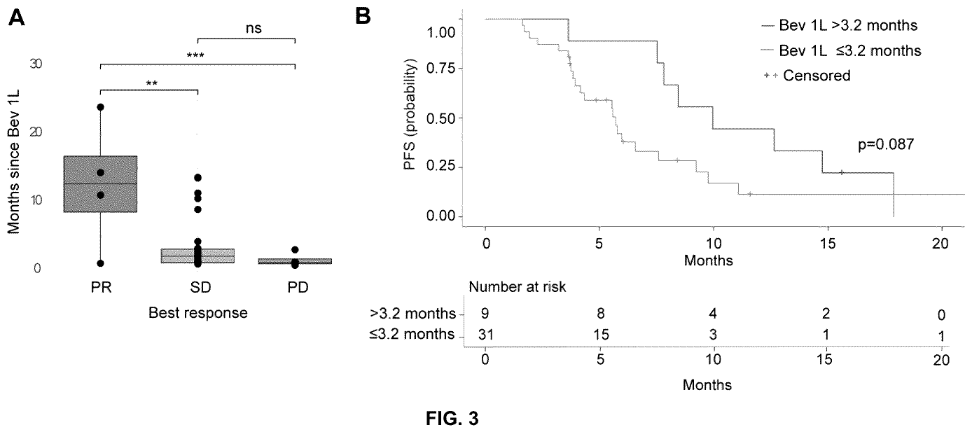

To address this question, the effect of time since bevacizumab first line (Bev 1L) on the clinical efficacy of onvansertib+FOLFIRI+bevacizumab in the 40 bevacizumab-exposed patients (TROV054 study) was assessed. The median time since Bev 1L was 2.0 months (IQR 1.0-3.2 months). Interestingly, patients achieving PR (n=4) had significantly longer time since Bev 1L compared to patients with SD (n=32) or PD (n=4) (panel A of FIG. 3), suggesting that sensitivity to onvansertib+FOLFIRI+bevacizumab may be restored following prolonged bevacizumab wash out period.

Additionally, the efficacy of the combination was compared in patients with long time since Bev 1L (>3.2 months, corresponding to the 3rd quartile of the IQR, n=9) versus patients with short since Bev 1L (≤3.2 months, n=31). Response to therapy was superior in patients with long time since Bev 1L with an ORR of 33% (95% CI 7-70, p=0.053) versus an ORR of 3% (95% CI 0-17, p=0.36) for patients with short time since Bev 1L. It was also observed a trend towards longer PFS in these patients with a median PFS of 10.0 months (95% CI, 7.8-NR) versus 5.7 months (95% CI, 4.2-9.2) in patients with short time since Bev 1L (panel B of FIG. 3). The data support that the resistance induced by bevacizumab can be reverted and that removal of bevacizumab treatment for a certain amount of time can resensitize tumors to onvansertib+FOLFIRI+bevacizumab.

### Example 3

### Time Effect on the Clinical Efficacy of Onvansertib+FOLFIRI+Bevacizumab

Similar to Example 2, this example further assesses the association between time since bevacizumab first-line treatment and efficacy of the combination of onvansertib, FOLFIRI and bevacizumab in the bev-exposed patients. Different from Example 2, wherein the analysis included only patients treated with onvansertib at 15 mg/m², this example expanded the analysis to include all patients evaluable for efficacy in the TROV054 study, incorporating those treated with onvansertib at 12 mg/m² and 18 mg/m².

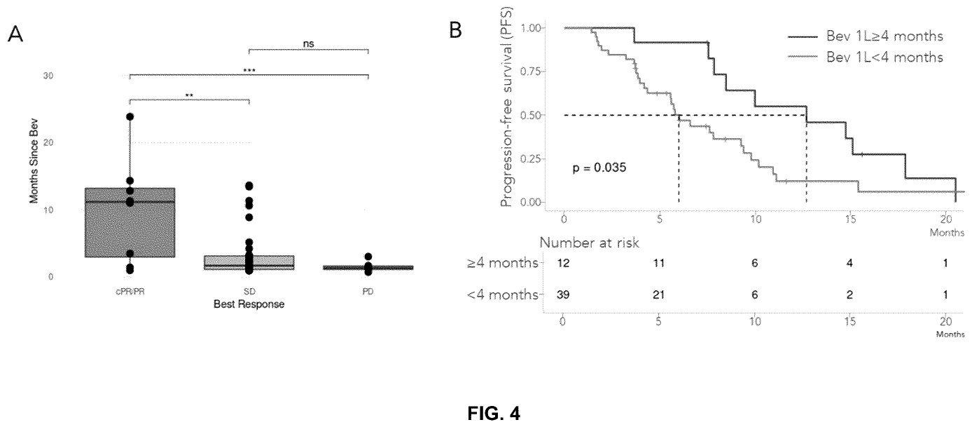

Of the 66 patients evaluable for efficacy across doses in TROV054, 51 had received Bev in 1L (Bev-exposed) with a median time since Bev 1L of 1.64 months (IQR: 1.1-3.3 months). Patients achieving PR (n=8) had significantly longer time since Bev 1L compared to patients with SD (n=37) or PD (n=6) (panel A of FIG. 4), supporting that sensitivity to onvansertib+FOLFIRI+bevacizumab may be restored following prolonged bevacizumab wash out period.

The ORR of the 51 Bev-exposed patients was 15.7%. Patients with long time since Bev 1L (>4 months, n=12) had significantly higher ORR of 42% ([95% CI: 15-72%], p=0.03), whereas patients with short time since Bev 1L (<4 months, n=39) had an ORR of 8% [95% CI: 2-21%](p=0.27). The patients were not exposed to Bev between 3.2 months to 4 months. Furthermore, patients with long time since Bev 1L had significantly longer PFS than patients with short time since Bev 1L (panel B of FIG. 4), with a median PFS of 12.7 months [95% CI: 8.4—not reached] versus 6.0 months [95% CI: 4.3-9.8].

### Example 4

### The PLK1 Inhibitor Onvansertib in Combination with FOLFIRI and Bevacizumab in Treatment Of KRAS-Mutant Metastatic Colorectal Cancer

This phase II study evaluated the efficacy and tolerability of onvansertib, a polo like kinase 1 (PLK1) inhibitor, in combination with fluorouracil, leucovorin, and irinotecan (FOLFIRI) 1 bevacizumab for the second-line treatment of KRAS-mutant metastatic colorectal cancer (mCRC). This multicenter, open-label, single-arm study enrolled KRAS-mutated mCRC patients previously treated with oxaliplatin and 5-FU with or without bevacizumab. Patients received 15 mg/m² onvansertib (on days 1-5 and 15-19 of a 28-day cycle) and FOLFIRI+bevacizumab (on days 1 and 15). The primary endpoint was the objective response rate (ORR), and secondary endpoints included progression-free survival (PFS), duration of response (DOR), and tolerability. Translational and preclinical studies were conducted in KRAS-mutant CRC.

The combination therapy was well-tolerated, achieving an objective response rate (ORR) of 26.4%, with post hoc analyses revealing significantly greater clinical benefit in in bevacizumab-naïve patients, who had an ORR of 76.9% compared to 10.0% in those previously exposed to bevacizumab.

Among the 53 patients treated, the confirmed ORR was 26.4% (95% CI, 15.3 to 40.3). The median DOR was 11.7 months (95% CI, 9.4 to not reached). Grade 3/4 adverse events were reported in 62% of patients. A post hoc analysis revealed that patients with no prior bevacizumab treatment had a significantly higher ORR and longer PFS compared to patients with prior bevacizumab treatment: ORR of 76.9% versus 10.0% (odds ratio of 30.0, p<0.001) and median PFS of 14.9 months versus 6.6 months (hazard ratio of 0.16, p<0.001). Translational studies suggested synergy between onvansertib and bevacizumab, while prior bevacizumab exposure appeared to contribute to treatment resistance. The translational findings also supported that prior bevacizumab exposure contributed to onvansertib resistance. Preclinically, it was shown that onvansertib inhibited the hypoxia pathway and exhibited robust antitumor activity in combination with bevacizumab through the inhibition of angiogenesis. The addition of a PLK 1 inhibitor to chemotherapy/bevacizumab exhibited very promising activity in patients with previously treated but bevacizumab-naïve KRAS-mutant mCRC.

Onvansertib in combination with FOLFIRI+bevacizumab showed significant activity in the second-line treatment of KRAS-mutant mCRC patients, particularly in patients with no prior bevacizumab treatment. These findings led to the evaluation of the combination in the first-line setting (ClinicalTrails.gov identifier: NCT06106308).

### INTRODUCTION

The mainstay of management for metastatic colorectal cancer (mCRC) patients harboring oncogenic KRAS mutations has not significantly changed in twenty years. Cytotoxic chemotherapy with fluoropyrimidines combined with either oxaliplatin or irinotecan, forms the backbone of treatment. The addition of antiangiogenic therapy, notably bevacizumab, a monoclonal antibody that targets vascular endothelial growth factor A (VEGFA), enhances chemotherapy efficacy in both first- and second-line treatments. However, the benefit of combining chemotherapy and antiangiogenic agents in the second-line setting remains limited, with an objective response rate (ORR) around 15% and a median progression-free survival (PFS) of 6 to 7 months.

Developing therapies targeting mutant KRAS (≈45% of CRC patients) has been challenging. Inhibitors of KRAS^{G12C}, a relatively rare mutation in CRC (≈3% of patients), have shown modest single agent activity, prompting the exploration of combination therapies. Most mCRC patients with KRAS mutations still lack effective targeted therapies, underscoring the urgent need for innovative treatments.

Polo-like kinase 1 (PLK1) has emerged as a promising target. The serine/threonine kinase PLK1 regulates the cell cycle by controlling mitotic entry and progression. Onvansertib, a selective PLK1 inhibitor, has shown potent antitumor activity in CRC preclinical models, both as a single agent and in combination with irinotecan. Additionally, synthetic lethality between PLK1 inhibition and KRAS mutation has been observed in CRC models, enhancing the therapeutic potential for KRAS-mutated patients. Onvansertib combined with FOLFIRI and bevacizumab was safe in KRAS-mutant mCRC patients, prompting further evaluation in phase II trials.

In this phase II study, the antitumor activity and tolerability of onvansertib at 15 mg/m² in combination with FOLFIRI and bevacizumab was assessed for the second-line treatment of KRAS-mutant mCRC patients. The observation of differential clinical benefit, dependent on prior bevacizumab exposure, prompted comprehensive translational analyses to understand the interplay between onvansertib and bevacizumab exposure. This study describes the prospective trial and subsequent translational analyses.

### Study Design and Patients

This study (ClinicalTrials.gov identifier: NCT03829410) was an open-label, single-arm phase Ib/II study conducted at 7 sites in the U.S., evaluating the efficacy and tolerability of onvansertib in combination with FOLFIRI and bevacizumab in second-line treatment of KRAS-mutant mCRC. The phase I portion described detailed eligibility criteria. Briefly, patients≥18-years-old with histologically confirmed metastatic and unresectable CRC, and a KRAS mutation in exons 2, 3 or 4 detected in the primary tumor or the metastasis by a CLIA-certified laboratory, were eligible. Patients were required to have received ≥6 weeks of oxaliplatin and fluoropyrimidine, with or without bevacizumab, and to have either progressed within 6 months of treatment or shown intolerance to oxaliplatin. Eastern Cooperative Oncology Group performance status (ECOG PS) of 0 or 1, and adequate organ function were required. Key exclusion criteria included microsatellite instability-high/mismatch repair deficiency, BRAF V600 mutation, more than one prior chemotherapy regimen for metastatic disease, and untreated brain metastasis.

### Treatment

Onvansertib was administrated orally at the recommended-phase 2 dose (RP2D) of 15 mg/m² on days 1-5 and 15-19 of a 28-day-cycle in combination with FOLFIRI (irinotecan 180 mg/m² intravenous (IV); leucovorin 400 mg/m² IV; fluorouracil (FU) 400 mg/m² IV bolus; FU 2400 mg/m² continuous IV infusion for 46 hours) and bevacizumab (5 mg/kg IV) administered on days 1 and 15 until disease progression, death, unacceptable toxicity, or withdrawal from the study. The elimination of the FU bolus and leucovorin was allowed at the investigator's discretion.

### Endpoints

The primary endpoint of the Phase II was the investigator-assessed ORR according to RECIST version 1.1 (v1.1) in patients who received at least 1 cycle (28 days) of treatment. Secondary efficacy endpoints included disease control rate (percentage of patients with best overall response being complete response (CR), partial response (PR) or stable disease (SD)), PFS (time from treatment initiation to progression or death, whichever occurs first), duration of response (DOR, time from first response (CR or PR) to progression or death, whichever occurred first), and reduction in KRAS-mutant allelic burden in liquid biopsies.

### Assessments

Tumor response was assessed using computed tomography or magnetic resonance imaging per RECIST v.1.1, at screening and during treatment at every other cycle until end-of-treatment. For patients who discontinued study treatment for reasons other than progressive disease, follow-up information was collected until the patient started new therapy, underwent a procedure, experienced progressive disease, or died, for up to one year.

Safety assessments in patients who received at least one treatment dose included physical examinations, laboratory test results, electrocardiograms, and monitoring of adverse events (AEs) graded by the National Cancer Institute Common Terminology Criteria for Adverse Events version 5.0.

Blood samples were collected at baseline and after completion of cycle 1 to analyze KRAS-mutant circulating tumor DNA (ctDNA) as described previously. For patients with available archival tumor tissue, the presence of KRAS mutations was tested using the Tempus xT assay (Tempus Labs Inc.).

### Tempus Real-World Patient Sample Selection and Analysis

Biopsies from KRAS-mutant mCRC patients were selected from the Tempus real-world database for gene expression analysis. Selected biopsies were from patients treated in first-line therapy with oxaliplatin with or without bevacizumab. Differential expression analysis and gene set enrichment analysis were performed as described below.

### Preclinical Studies

In vitro and in vivo studies were performed using the human KRAS-mutant CRC cell lines HCT116 (KRAS G13D), SW620 (KRAS G12V), LoVo (KRAS G13D) and DLD-1 (KRAS G13D) as described below.

### Statistical Analyses

The study employed a binomial superiority one-sample test with a null hypothesis set at an ORR of 15%, and an alternative hypothesis at an ORR of 30%. The sample size was estimated at approximately 80 patients to obtain 85% power with a Type I error of 2.5%. Patient characteristics and AEs were summarized using descriptive statistics. PFS and DOR were estimated using the Kaplan-Meier method with 95% confidence intervals (CI). Post hoc univariate and multivariable analyses were used to determine the impact of baseline characteristics on ORR by a logistic regression model and on PFS by the Cox proportional hazards regression model. Variables with p-values<0.05 in univariate analyses were set as covariates in the multivariable analyses.

One-way ANOVA with Dunnet's multiple comparison test was used for analyses of preclinical studies, p-values<0.05 were considered statistically significant. Analyses were performed using R version 4.2.3 and GraphPad Prism version 10.0.02.

### Tempus Real-World Patient Sample Selection and Analysis

Biopsies were obtained within a time frame of 6 weeks to one year after initiating first-line treatment and prior starting second-line therapy. Differential expression analysis was performed using a Wilcoxon rank-sum test and a linear model controlling for tumor purity. Genes were ranked by the product of the Log 10 transformed p-value and fold change. Differential expression results were then used for gene set enrichment analysis (GSEA) using the fgsea package. Gene sets were selected from the Molecular Signatures Database (MSigDB) including MSigDB hallmark and oncogenic signature gene sets.

### Cell Culture and Transfection

Human KRAS-mutant colorectal cancer cell lines HCT116, SW620, LoVo and DLD-1 (ATCC) were cultured in RPMI (Cat #30-2001, ATCC) supplemented with 10% FBS (Cat #16000069, ThermoFisher Scientific) and 1× penicillin-streptomycin solution (Cat #30-2300, ATCC). The cells were incubated at 37° C. in a humidified chamber containing 5% CO₂ and 20% O₂. Hypoxic conditions were achieved by culturing cells in Ruskinn-Baker InvivO₂ 400 workstation (SP0530) at 1% 02 and 5% CO₂. The cell lines were confirmed negative for Mycoplasma using MycoAlert Mycoplasma Detection kit (Cat #LT07-318, Lonza, Basel, Switzerland).

For gene silencing experiments, cells were transiently transfected with 50 nM of SMARTpool siRNA containing a mixture of 4 siRNAs (ON-TARGETplus siRNA, Horizon Discovery) targeting PLK1 (Cat #L-003290-00-0005), using Lipofectamine™ RNAiMAX transfection reagent (Cat #13778075, ThermoFisher Scientific) following the manufacturer's protocol. The ON-TARGETplus non-targeting siRNA pool (Cat #D-001810-10-05) was used as the control.

### Protein Analysis

Cells lysates were prepared in ice-cold lysis buffer (10 mM Tris-HCl (pH 7.4), 25 mM β-glycerophosphate, 150 mM NaCl, 20 mM NaF, 1 mM Na₃VO₄, 5 mM EDTA, 1 mM Na₄P₂O₇, 10% glycerol, 1% IGEPAL CA-630) supplemented with protease inhibitor cocktail (Cat #P8340, Millipore Sigma) and phosphatase inhibitor cocktail (Cat #P5726, Millipore sigma). Total protein concentration was determined using the Pierce BCA Protein Assay Kit (Cat #23227, ThermoFisher Scientific) according to the manufacturer's instructions. Protein lysates were analyzed by immunoblot using the Jess Simple Western instrument (Bio-Techne) as per manufacturer's instructions and described previously. The following primary and secondary antibodies were used: PLK1 (Cat #4513, dilution 1:200, Cell Signaling Technology), HIF1α (Cat #ab179483, dilution 1:100, Abcam), β-Actin-HRP (Cat #sc-47778, dilution 1:500, Santa Cruz), and anti-rabbit secondary HRP (Cat #042-206, dilution 1:1, Bio-Techne). Data analysis was performed using Compass software for Simple Western.

### RNA Extraction

Cells were lysed in 700 L of Trizol LS Reagent (Cat #10296028, ThermoFisher Scientific) and total RNA was isolated using the PureLink™ RNA Mini Kit (Cat #12183025, ThermoFisher Scientific) per the manufacturer's instructions.

### Real-Time Quantitative Polymerase Chain Reaction (RT-qPCR)

The concentration and purity of RNA were determined using a NanoDrop 2000C spectrophotometer (ThermoFisher Scientific). The reverse transcription of RNA was performed using Superscript IV VILO Master Mix (Cat #11766050, ThermoFisher Scientific). RT-qPCR was performed in triplicates with Primetime gene expression master mix (Cat #1055771, Integrated DNA Technologies) on a CFX384 thermocycler (Bio-Rad). Primers were purchased from Integrated DNA Technologies, and primer reference numbers are provided in table 1 below. The housekeeping gene RPLP0 was used to normalize expression. Relative expression values were calculated using the comparative cycle threshold method (ΔΔCt).

TABLE 1
List of primers used for RT-qPCR

| Gene | Assay ID |
|---|---|
| ADM | Hs.PT.56a.25211580.g |
| VEGFA | Hs.PT.58.1149801 |
| ANGPTL4 | Hs.PT.58.25480012 |
| CA9 | Hs.PT.56a.2192889 |
| PDK1 | Hs.PT.58.19794808 |
| SLC2A3 | Hs.PT.58.39033300 |
| DDIT4 | Hs.PT.58.38843854.g |
| BNIP3L | Hs.PT.56a.1103799 |
| NDRG1 | Hs.PT.58.4224283 |
| RPLP0 | Hs.PT.39a.22214824 |

### RNA-Seq Library Generation, High-Throughput Sequencing, and Analysis

For RNA-sequencing, biological triplicate samples were analyzed. Total RNA samples were quantified using Qubit 2.0 Fluorometer (Life Technologies) and RNA integrity was checked with the TapeStation 4200 (Agilent Technologies). RNA sequencing libraries were prepared using the NEBNext Ultra II RNA Library Prep Kit for Illumina per the manufacturer's instructions (Cat #:E7775, New England Biolabs). Briefly, mRNAs were initially enriched with Oligo d(T) beads. Enriched mRNAs were fragmented for 15 minutes at 94° C. First strand and second strand cDNA were subsequently synthesized. cDNA fragments were end repaired and adenylated at 3′ ends, and universal adapters were ligated to cDNA fragments, followed by index addition and library enrichment by PCR with limited cycles. The sequencing library was validated on the Agilent TapeStation, and quantified by using Qubit 2.0 Fluorometer as well as by quantitative PCR (KAPA Biosystems).

The sequencing libraries were multiplexed and clustered onto a flow cell on the Illumina NovaSeq instrument according to manufacturer's instructions. The samples were sequenced using a 2×150 bp Paired End configuration. Image analysis and base calling were conducted by the NovaSeq Control Software. Raw sequence data (.bcl files) generated from Illumina NovaSeq was converted into fastq files and de-multiplexed using Illumina bcl2fastq software (version 2.20). One mismatch was allowed for index sequence identification.

Gene expression was quantified using Salmon v1.9.0 with GRCh38 (GENCODE release 44). Differential gene expression was assessed using DESeq2 (version 1.42.1). Log 2 fold-changes (LFC) were shrunken using the ashr algorithm and p-values were adjusted using independent hypothesis weighting against the ‘baseMean’ counts and the Benjamini-Hochberg method.

For each comparison (hypoxia DMSO versus normoxia DMSO and hypoxia onvansertib versus hypoxia DMSO), genes were ranked by their LFC and GSEA carried out against the Hallmarks of Cancer gene set collection from MSigDB (version 2023) using fGSEA (version 1.28.0).

Genes that were differentially regulated in response to both exposure to hypoxic conditions and treatment with onvansertib in both cell lines were selected. The variance transformed (rlog) gene counts for these genes in each cell and contrast were z-scaled and clustered, using complete linkage and the Euclidean distance, and plotted as a heat map using the ComplexHeatmap R package (version 2.18.0).

### In Vivo Studies

All animal studies were reviewed and approved by the Institutional Animal Care and Use Committee of Crown Bioscience prior to being carried out. The care and use of animals were conducted in accordance with the regulations of the Association for Assessment and Accreditation of Laboratory Animal Care. Female Balb/c nude mice (6-9 weeks, GemPharmatech Co. Ltd) were inoculated subcutaneously with HCT116, SW620 or LoVo cells. Once tumors reached 100-200 mm³, mice were randomized and treated with vehicle, onvansertib (oral, 45 mg/kg, daily), bevacizumab (intraperitoneally, 5 mg/kg for SW620 and HCT116, 1 mg/kg for LoVo, twice a week) or the combination. Onvansertib (provided by Cardiff Oncology) was prepared as described previously and bevacizumab (HY-P9906, MedChemExpress) was diluted in saline. Tumor volumes and body weights were measured twice a week. Tumors collected at end of study were subjected to gross morphology and histology analyses.

### Histopathology Analyses

Tumor samples were collected at the end of study and fixed for 24 hours in 10% neutral buffered formalin for immunohistochemical (IHC) and hematoxylin and eosin (H&E) staining. BrdU (2 mg/mice, Cat #HY-15910, MedChemExpress) was administered to the mice 24 hours before tumor sample collection. Fixed tumor tissues were paraffinembedded, sectioned and placed on positively charged glass slides. Sections were deparaffinized and stained with anti-CD31 (Cat #ab28364, dilution 1:50, Abcam) or anti-BrdU (Cat #ab6326, dilution 1:600, Abcam) antibodies for 1 hour at 37° C. using a Leica Bond III autostainer with Leica detection kit (Cat #DS9800, Leica Biosystems). Brightfield whole slide imaging was performed using Aperio AT2 scanner (Leica Biosystems).

The digital images were examined by a board-certified pathologist to quantify apoptotic cells and CD31-positive vessels. For each sample, tumor cells exhibiting apoptotic features were manually counted across five randomly selected fields of view at 50 m magnification. CD31-positive vessels were identified as round/oval CD31-positive endothelial layer within the vascular wall and counted in five random fields per tumor at 100 m magnification. The number of BrdU-positive tumor cells and the intensity of the stain were quantified using Halo AI classifier (Indica Labs, version 3.6). Stain intensity was scored as negative, weak, moderate or strong, and an H-score was calculated for each sample as follows:

$$[ ( 0 \times \% negative cells ) + ( 1 \times \% weak positive cells ) \text{} + \backslash [ AutoLeftMatch ] ( 2 \times \% moderate positive cells ) \backslash [ NoBreak ] + ( 3 \times \% strong positive cells ) ] .$$

### Results

### Patients

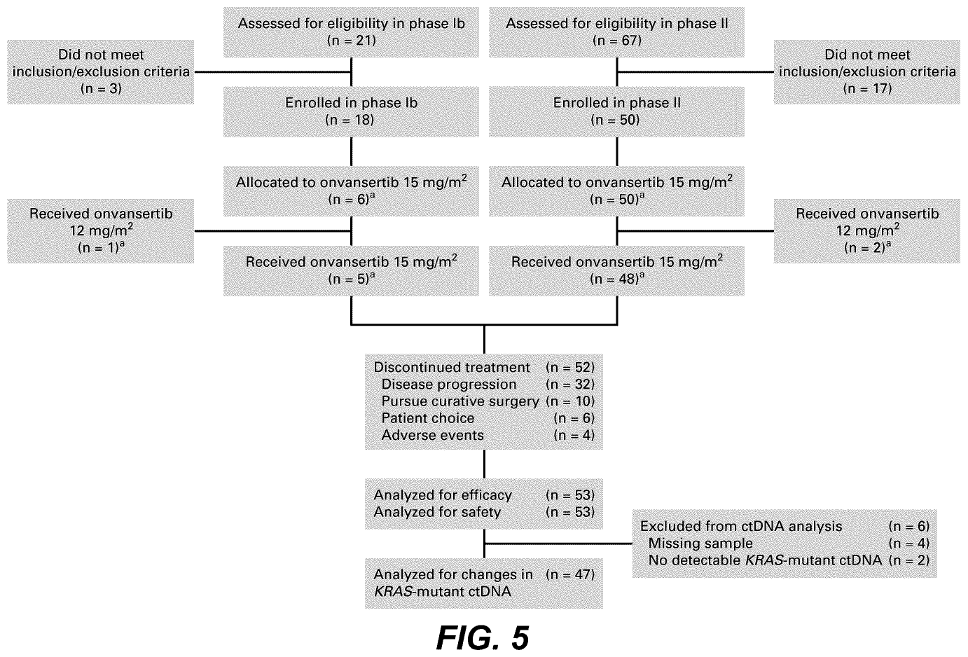

Between December 2019 and October 2023, 53 patients were treated with onvansertib at 15 mg/m², including 5 patients treated in Phase Ib (FIG. 5). Patient baseline demographics and disease characteristics are summarized in Table 2. As of Jan. 29, 2024, all patients had completed their on-treatment visits and follow-up period, and median follow-up duration was 6.4 months (range, 1.6 to 30.3). The reasons for discontinuing treatment were disease progression (n=32, 60.4%), pursued curative surgery (n=10, 18.9%), AEs (n=4, 7.5%), and patient choice (n=6, 11.3%). One patient (1.9%) enrolled in an expanded access program to continue receiving onvansertib following the study's closure.

TABLE 2
Baseline demographics and disease characteristics

| Characteristic | Patients (n = 53) | |
|---|---|---|
| Median age (range), years | 54 | (34-83) |
| Gender, n (%) | | |
| Male | 27 | (50.9) |
| Female | 26 | (49.1) |
| Race, n (%) | | |
| White | 44 | (83.0) |
| Other | 9 | (17.0) |
| ECOG, n (%) | | |
| 0 | 29 | (54.7) |
| 1 | 24 | (45.3) |
| Primary tumor site, n (%) | | |
| Left colon or rectum | 34 | (64.1) |
| Right colon | 18 | (34.0) |
| Unknown | 1 | (1.9) |
| Metastatic disease at diagnosis, n (%) | | |
| Yes | 44 | (83.0) |
| No | 9 | (17.0) |
| Liver metastasis, n (%) | | |
| Yes | 39 | (73.6) |
| No | 14 | (26.4) |
| Metastatic lesions, n (%) | | |
| Single | 3 | (5.7) |
| Multiple | 50 | (94.3) |
| Metastatic organs, n (%) | | |
| Single | 14 | (26.4) |
| Multiple | 39 | (73.6) |
| Received prior bevacizumab, n (%) | | |
| Yes | 40 | (75.5) |
| No | 13 | (24.5) |

### KRAS Mutations

The frequency of KRAS mutations in this study aligned with the expected distribution for this patient population (Table 3). The presence of a KRAS mutation was confirmed for all patients through molecular testing of plasma ctDNA and/or archival tumor tissue (Table 4).

TABLE 3
Frequency of KRAS Mutations

| KRAS Mutation | No. of Patients | % |
|---|---|---|
| G12D | 16 | 30 |
| G12V | 10 | 19 |
| G12A | 6 | 11 |
| G13D | 6 | 11 |
| G12C | 4 | 8 |
| A146T | 3 | 6 |
| Q61H | 3 | 6 |
| G12S | 2 | 4 |
| K117N | 2 | 4 |
| G12R | 1 | 2 |
| Total | 53 | 100 |

TABLE 4
List of KRAS Mutations Detected in Liquid and Tumor Biopsies

| | KRAS Mutation | KRAS Mutation |
|---|---|---|
| | Detected in | Detected in |
| Patient | Liquid Biopsy | Archival Tissue |
| 1 | G12A | G12A |
| 2 | G12D | NA |
| 3 | A146T | NA |
| 4 | G12D | G12D |
| 5 | G12V | NA |
| 6 | G13D | NA |
| 7 | G12D | NA |
| 8 | G12A | NA |
| 9 | G12D | NA |
| 10 | G12D | G12D |
| 11 | G12D | G12D |
| 12 | G12A | G12A |
| 13 | G12D | NA |
| 14 | Not detected | Q61H |
| 15 | K117N | K117N |
| 16 | Q61H | NA |
| 17 | A146T | NA |
| 18 | G12A | G12A |
| 19 | Not detected | Q61H |
| 20 | G12V | G12V |
| 21 | G12V | NA |
| 22 | G12D | G12D |
| 23 | G12D | NA |
| 24 | G12A | G12A |
| 25 | G12C | G12C |
| 26 | G13D | G13D |
| 27 | G12S | G12S |
| 28 | G13D | Not detected |
| 29 | G12D | G12D |
| 30 | G13D | NA |
| 31 | G12C | NA |
| 32 | G12D | NA |
| 33 | G12V | NA |
| 34 | G12D | G12D |
| 35 | G12R | G12R |
| 36 | G12D | G12D |
| 37 | G12S | NA |
| 38 | G12V | G12V |
| 39 | G12A | G12A |
| 40 | G12C | G12C |
| 41 | G12V | NA |
| 42 | K117N | K117N |
| 43 | G12V | G12V |
| 44 | G12D | G12D |
| 45 | A146T | NA |
| 46 | G12V | G12V |
| 47 | G13D | G13D |
| 48 | G12V | G12V |
| 49 | G12A | G12A |
| 50 | G12D | G12D |
| 51 | G13D | G13D |
| 52 | G12V | G12V |
| 53 | G12D | G12D |

Abbreviation: NA, no data available

### Clinical Efficacy

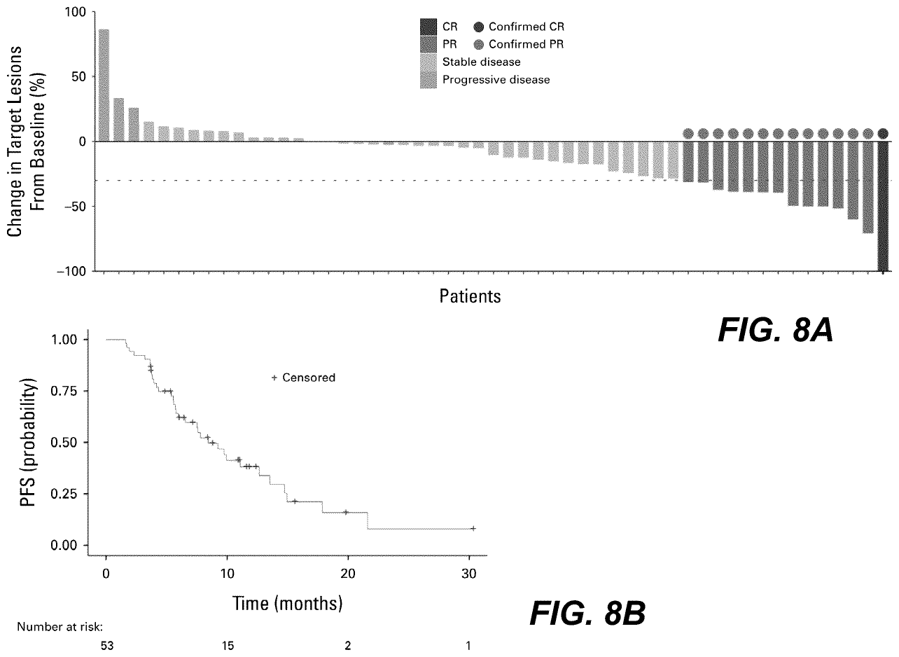

All patients treated with onvansertib RP2D completed at least one cycle of treatment and were included in the efficacy analysis (FIG. 5). The investigator-assessed ORR was 26.4% (95% CI, 15.3 to 40.3), including 1 CR and 13 PR, and all responses were confirmed (FIG. 8A-FIG. 8B). Seven (500%) of the responders achieved PR at their first disease evaluation scan (2 cycles of treatment). Median time to achieve a response was 86 days. Thirty-nine (73.60%) patients had a decrease in target lesion size from baseline and the disease control rate, including CR, PR, and SD, was 92.5% (49/53 patients). The median duration of response was 11.7 months (9500 CI, 9.4 to not reached) and the median PFS was 8.4 months (95% CI, 6.0 to 14.8) (FIG. 8A-FIG. 8B).

Circulating Tumor DNA (ctDNA)

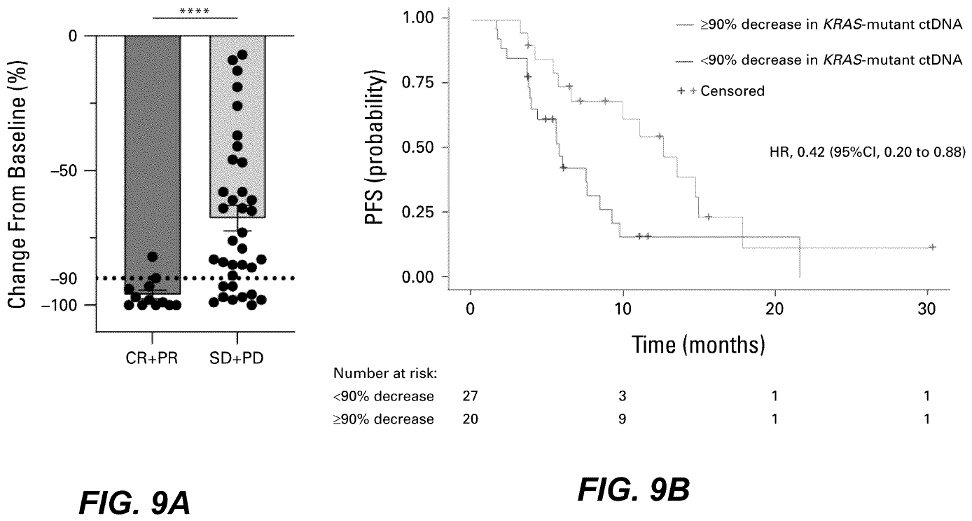

It was previously described that a ≥90% decrease in KRAS-mutant ctDNA after 1 cycle of treatment was associated with clinical benefit to onvansertib+FOLFIRI and bevacizumab. Here, patients achieving CR/PR (n=12) had a significantly greater decrease in KRAS-mutant ctDNA compared with those with SD or PD (n=35, FIG. 9A-FIG. 9B). Twenty patients had a ≥90% decrease in KRAS-mutant ctDNA, and when compared to those with <90% reduction (n=27), exhibited a significantly higher ORR of 55.0% (95% CI, 31.5 to 76.9) versus 3.7% (95% CI, 0.1 to 19.0) and a significantly longer median PFS of 12.6 months (95% CI, 10.0 to not reached) versus 5.8 (95% CI, 3.9 to 9.2; HR=0.42, p=0.019) (FIG. 9A-FIG. 9B).

### Safety

Treatment-emergent adverse events (TEAEs) of any grade were reported in all patients with the most common being fatigue (73.6%), neutropenia (71.7%), nausea (62.3%), diarrhea (52.8%) and stomatitis (45.3%) (Table 5). Grade 3 and 4 TEAEs were reported in 33 (62.3%) and 4 (7.5%) patients, respectively. The grade 4 TEAEs were neutropenia in 3 patients (5.7%) and colonic perforation in one patient (1.9%). The most common grade 3 TEAEs were neutropenia in 19 patients (35.8%), hypertension in 5 patients (9.4%), diarrhea and nausea in 4 patients (7.5%) each. No neutropenic fever was reported. Four patients discontinued treatment due to an AE, but none were related to onvansertib.

TABLE 5
Summary of Treatment-Related Adverse Events Reported in ≥10% of Patients

| | No. of Patients With the Event,^{a} No. (%) | | | | | | | | | |
|---|---|---|---|---|---|---|---|---|---|---|
| Adverse Event | Any Grade | | Grade 1 | | Grade 2 | | Grade 3 | | Grade 4 | |
| Any adverse events | 53 | (100) | 51 | (96.2) | 47 | (88.7) | 33 | (62.3) | 4 | (7.5)^{b} |
| Fatigue | 39 | (73.6) | 24 | (45.3) | 12 | (22.6) | 3 | (5.7) | 0 | |
| Neutropenia | 38 | (71.7) | 1 | (1.9) | 15 | (28.3) | 19 | (35.8) | 3 | (5.7) |

| Nausea | 33 | (62.3) | 21 | (39.6) | 8 | (15.1) | 4 | (7.5) | 0 |
|---|---|---|---|---|---|---|---|---|---|
| Diarrhea | 28 | (52.8) | 14 | (26.4) | 10 | (18.9) | 4 | (7.5) | 0 |
| Stomatitis | 24 | (45.3) | 14 | (26.4) | 7 | (13.2) | 3 | (5.7) | 0 |
| Alopecia | 17 | (32.1) | 13 | (24.5) | 4 | (7.5) | 0 | | 0 |
| Leukopenia | 17 | (32.1) | 4 | (7.5) | 10 | (18.9) | 3 | (5.7) | 0 |
| Vomiting | 16 | (30.2) | 8 | (15.1) | 5 | (9.4) | 3 | (5.7) | 0 |
| Anemia | 15 | (28.3) | 13 | (24.5) | 1 | (1.9) | 1 | (1.9) | 0 |
| Thrombocytopenia | 15 | (28.3) | 12 | (22.6) | 3 | (5.7) | 0 | | 0 |
| Constipation | 11 | (20.8) | 10 | (18.9) | 1 | (1.9) | 0 | | 0 |
| Dehydration | 10 | (18.9) | 3 | (5.7) | 4 | (7.5) | 3 | (5.7) | 0 |
| Hypertension | 10 | (18.9) | 2 | (3.8) | 3 | (5.7) | 5 | (9.4) | 0 |
| Abdominal pain | 8 | (15.1) | 6 | (11.3) | 1 | (1.9) | 1 | (1.9) | 0 |

| Decreased appetite | 8 | (15.1) | 4 | (7.5) | 4 | (7.5) | 0 | 0 |
|---|---|---|---|---|---|---|---|---|
| Epistaxis | 8 | (15.1) | 8 | (15.1) | 0 | | 0 | 0 |
| Palmar-plantar erythrodysesthesia | 7 | (13.2) | 5 | (9.4) | 2 | (3.8) | 0 | 0 |
| syndrome | | | | | | | | |
| Aspartate aminotransferase | 6 | (11.3) | 4 | (7.5) | 2 | (3.8) | 0 | 0 |
| increased | | | | | | | | |

| | | | | | | | |
|---|---|---|---|---|---|---|---|
| Lymphopenia | 6 | (11.3) | 0 | 6 | (11.3) | 0 | 0 |

^{a}If a patient experienced an adverse event multiple times, the maximum grade is counted.
^{b}Grade 4 colonic perforation was reported in one patient.

### Association Between Prior Bevacizumab Treatment and Clinical Bene Fit

In a post hoc analysis, the association between clinical benefit and baseline demographics or disease characteristics was evaluated. Univariate analyses for radiographic response and PFS indicated superior clinical benefit for patients who had not received bevacizumab as part of their first-line treatment (bevacizumab-naïve) and for patients without metastatic disease at time of diagnosis (Table 6). Multivariable analyses were carried out with these two characteristics to adjust for potential confounding effects on treatment outcomes. Only the absence of prior bevacizumab treatment remained independently associated with clinical benefit (Table 6). Bevacizumab-naïve patients (n=13) had an ORR of 76.9% (95% CI, 46.2 to 95.0) and a median PFS of 14.9 months (95% CI, 13.5 to not reached), compared to an ORR of 10.0% (95% CI, 2.8 to 23.7) and a median PFS of 6.6 months (95% CI, 5.6 to 9.8) for patients who received bevacizumab in first-line (bevacizumab-exposed, n=40) (FIG. 2A-FIG. 2B). The study was ended early based on the result of this subgroup analysis, showing significantly higher clinical benefit in bevacizumab-naïve patients.

TABLE 6
Univariate and multivariate analyses for radiographic response and PFS

| | Univariate Analyses | | | | Multivariable Analyses | | | |
|---|---|---|---|---|---|---|---|---|
| | Radiographic Response | | PFS | | Radiographic Response | | PFS | |
| Parameter | OR (95% CI) | P | HR (95% CI) | P | OR (95% CI) | P | HR (95% CI) | P |
| Age, years: | 0.92 | 0.946 | 1.64 | 0.449 | | | | |
| >70 v ≤70 | (0.04 to 7.97) | | (0.49 to 5.44) | | | | | |
| Sex: | 0.64 | 0.480 | 0.80 | 0.532 | | | | |
| Male v female | (0.18 to 2.19) | | (0.40 to 1.60) | | | | | |
| Race: | 2.72 | 0.196 | 0.86 | 0.745 | | | | |
| Other v White | (0.58 to 12.3) | | (0.35 to 2.11) | | | | | |
| ECOG PS: | 0.38 | 0.137 | 1.08 | 0.827 | | | | |
| 1 v 0 | (0.09 to 1.35) | | (0.55 to 2.13) | | | | | |
| Primary tumor site: | 0.99 | 0.990 | 2.07 | 0.065 | | | | |
| Right colon/other v | (0.26 to 3.48) | | (0.98 to 4.37) | | | | | |
| left colon/rectum | | | | | | | | |
| Metastatic disease | 0.11 | 0.005 | 3.28 | 0.022 | 0.59 | 0.636 | 0.82 | 0.795 |
| at diagnosis: | (0.02 to 0.51) | | (1.00 to 10.8) | | (0.07 to 6.16) | | (0.19 to 3.60) | |
| Yes v no | | | | | | | | |
| Liver metastasis: | 0.86 | 0.832 | 1.53 | 0.269 | | | | |
| Yes v no | (0.23 to 3.69) | | (0.70 to 3.34) | | | | | |
| Metastatic lesions: | 0.70 | 0.785 | 2.00 | 0.446 | | | | |
| Multiple v single | (0.06 to 15.9) | | (0.27 to 14.8) | | | | | |
| Metastatic organs: | 0.54 | 0.367 | 1.69 | 0.200 | | | | |
| Multiple v single | (0.14 to 2.13) | | (0.73 to 3.92) | | | | | |
| Received prior | 30.0 | <.001 | 0.16 | <.001 | 23.7 | <.001 | 0.14 | 0.002 |
| bevacizumab: | (6.5 to 187.6) | | (0.05 to 0.51) | | (4.2 to 201.0) | | (0.03 to 0.61) | |
| No v yes | | | | | | | | |

NOTE.
Bold indicates statistically significant association between the variable of interest and the outcome.
Abbreviations: ECOG PS, Eastern Cooperative Oncology Group performance status; HR, hazard ratio; OR, odds ratio; PFS, progression-free survival.

Transcriptomic Changes Associated with Bevacizumab Treatment

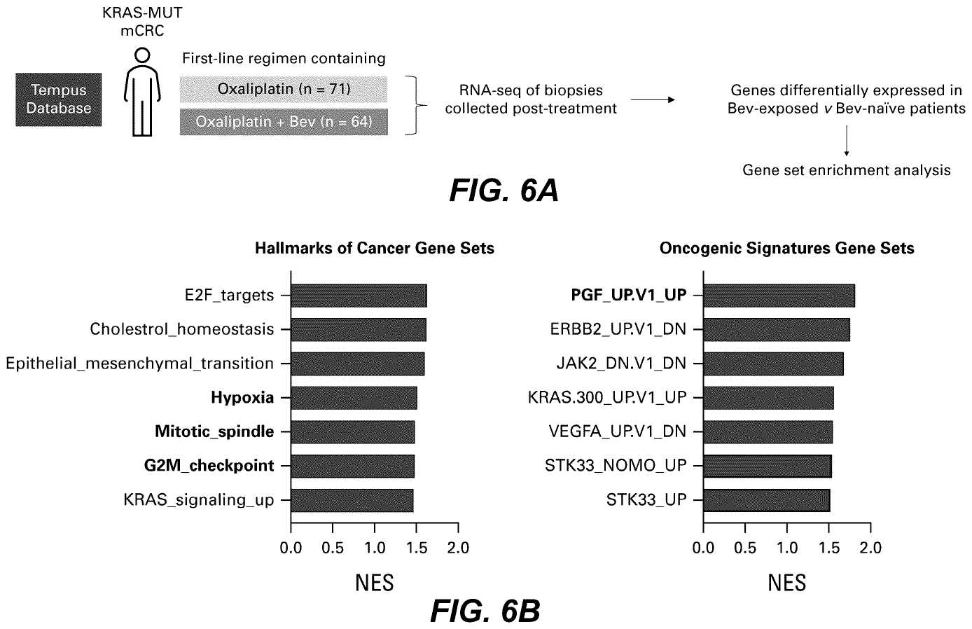

Potential resistance mechanisms arising from bevacizumab treatment was then investigated. The Tempus real-world database was leveraged and tumor transcriptomes obtained from KRAS-mutant mCRC patients after first-line treatment were compared with either oxaliplatin (bevacizumab-naïve, n=71) or oxaliplatin+bevacizumab (b evacizumabexposed, n=64), as depicted in FIG. 6A. Patient baseline characteristics were well balanced between these two cohorts (Table 7). Using gene set enrichment analysis (GSEA) on the MSigDB Oncogenic Signature and Hallmarks of Cancer gene sets, pathways upregulated in bevacizumab-exposed tumors were identified (FIG. 6B). These included pathways associated with bevacizumab resistance, such as the hallmark “hypoxia” and the oncogenic signature related to the angiogenic factor Placental Growth Factor (PGF). Furthermore, this analysis revealed upregulation of the hallmarks “G2M checkpoint” and “mitotic spindle” in bevacizumab-exposed tumors, two pathways linked to PLK1's functions in mitosis, and potential contributors of onvansertib resistance. Collectively, these data suggested that transcriptomic changes following bevacizumab exposure might drive resistance to both bevacizumab and onvansertib.

TABLE 7
Patient Demographics and Baseline Characteristics
of the Tempus Cohorts

| | | | |
|---|---|---|---|
| Characteristic | Bev-Naive | Bev-Exposed | P |

| Patients, No. | 71 | | 64 | | |
|---|---|---|---|---|---|
| Mean age at biopsy | 55.6 | | 55 | | 0.774 |
| collection, years | | | | | |
| Sex, No. (%) | | | | | 1.000 |
| Female | 36 | (50.7) | 33 | (51.6) | |
| Male | 35 | (49.3) | 31 | (48.4) | |
| Ethnicity, No. (%) | | | | | 0.062 |
| White | 30 | (42.3) | 32 | (50.0) | |
| Other | 19 | (26.8) | 6 | (9.4) | |
| Unknown | 22 | (31.0) | 26 | (40.6) | |
| ECOG PS, No. (%) | | | | | 0.218 |
| 0 | 28 | (39.4) | 20 | (31.3) | |
| 1 | 29 | (40.8) | 28 | (43.8) | |
| >1 | 5 | (7.0) | 2 | (3.1) | |
| Unknown | 9 | (12.7) | 14 | (21.9) | |
| Biopsy site, No. (%) | | | | | 0.992 |
| Liver | 26 | (36.6) | 25 | (39.1) | |
| Lower GI | 23 | (32.4) | 20 | (31.2) | |
| Pelvic and upper body | 10 | (14.1) | 9 | (14.1) | |
| soft tissue | | | | | |
| Other | 12 | (16.9) | 10 | (15.6) | |
| Metastatic disease at | | | | | 0.157 |
| diagnosis, No. (%) | | | | | |
| Yes | 53 | (74.6) | 39 | (60.9) | |
| No | 14 | (19.7) | 22 | (34.4) | |
| Unknown | 4 | (5.6) | 3 | (4.7) | |
| KRAS mutation | | | | | 0.665 |
| G12D | 21 | (30) | 19 | (30) | |
| G12V | 15 | (21) | 14 | (22) | |
| G13D | 11 | (15) | 6 | (9) | |
| G12C | 8 | (11) | 5 | (8) | |
| Other | 16 | (23) | 20 | (31) | |

Note:
P values are based on x2 test for categorical variables and t test for continuous variables.
Abbreviations: Bev, bevacizumab; ECOG PS, Eastern Cooperative Oncology Group performance status.

### Onvansertib and Bevacizumab Antitumor Activity in KRAS-Mutant CRC Xenografts

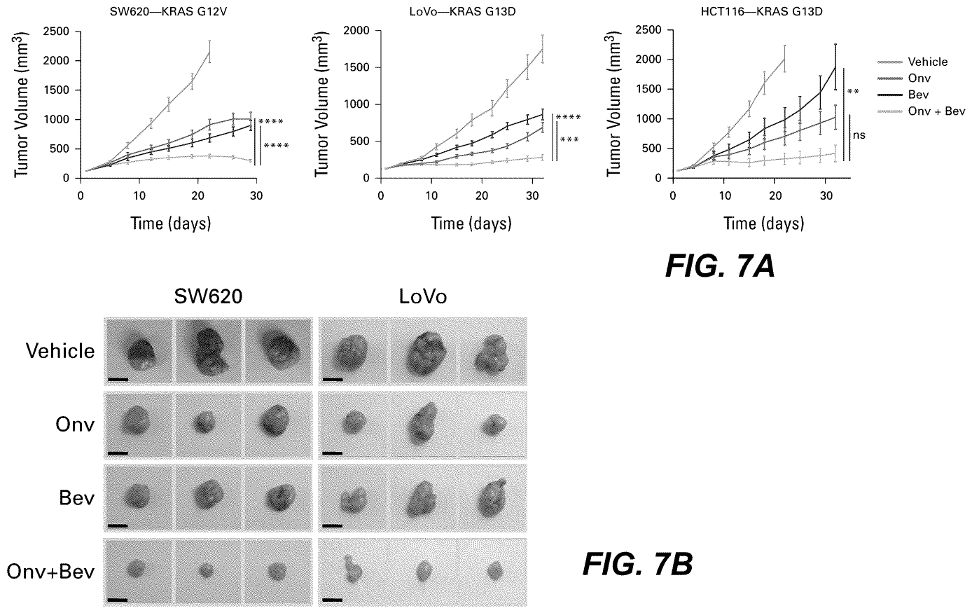

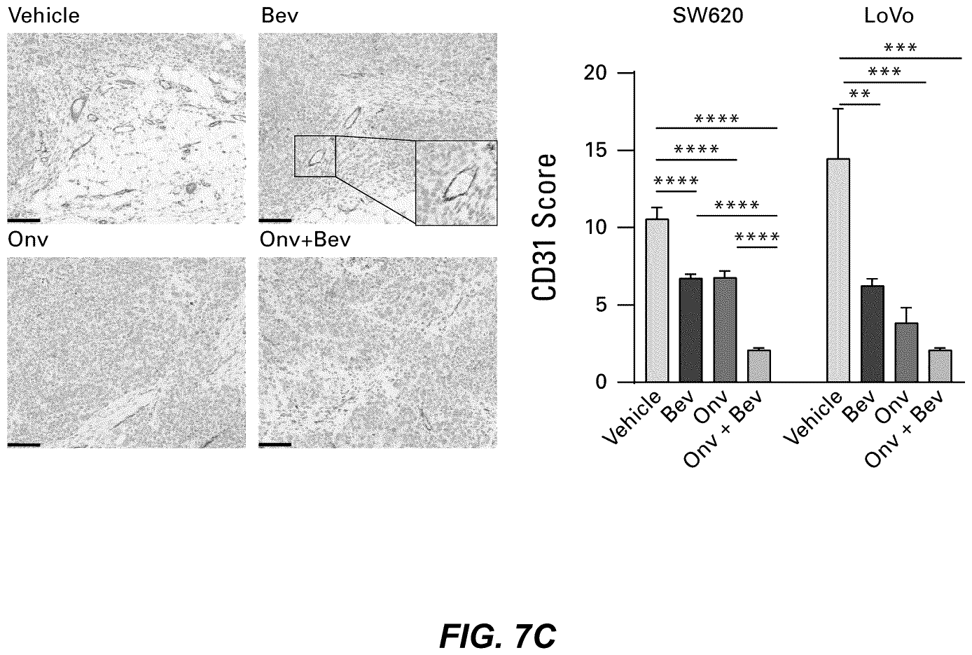

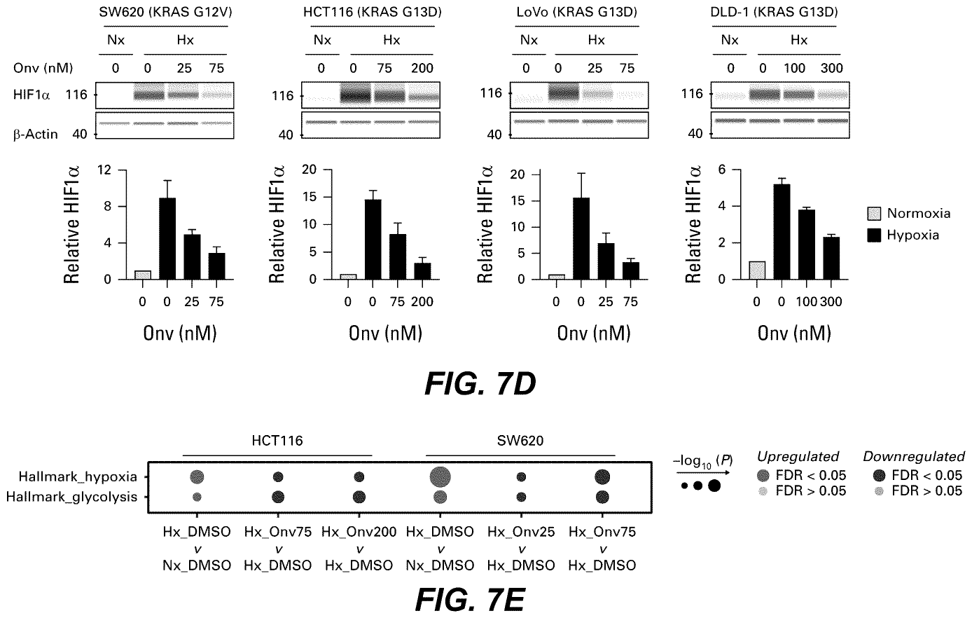

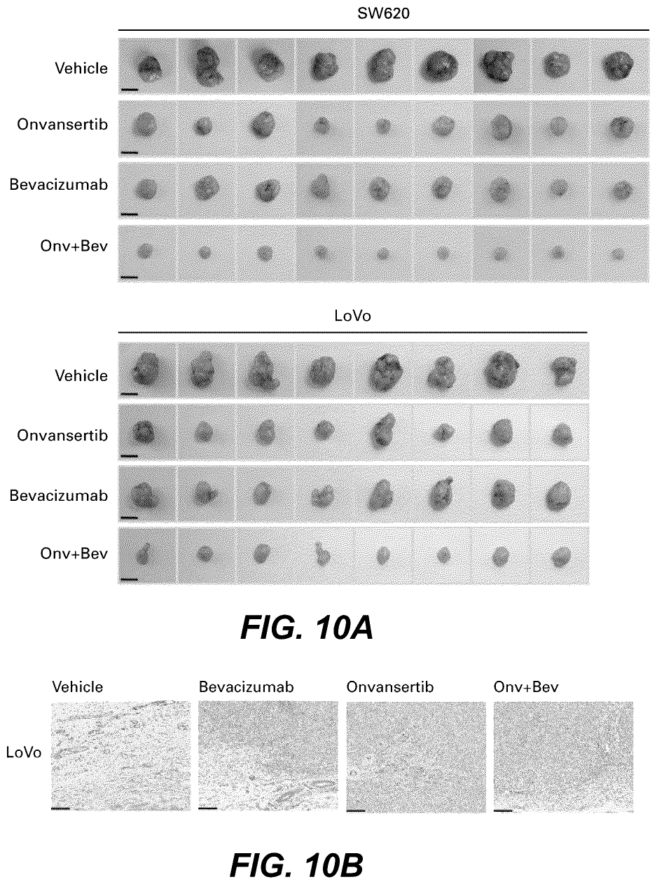

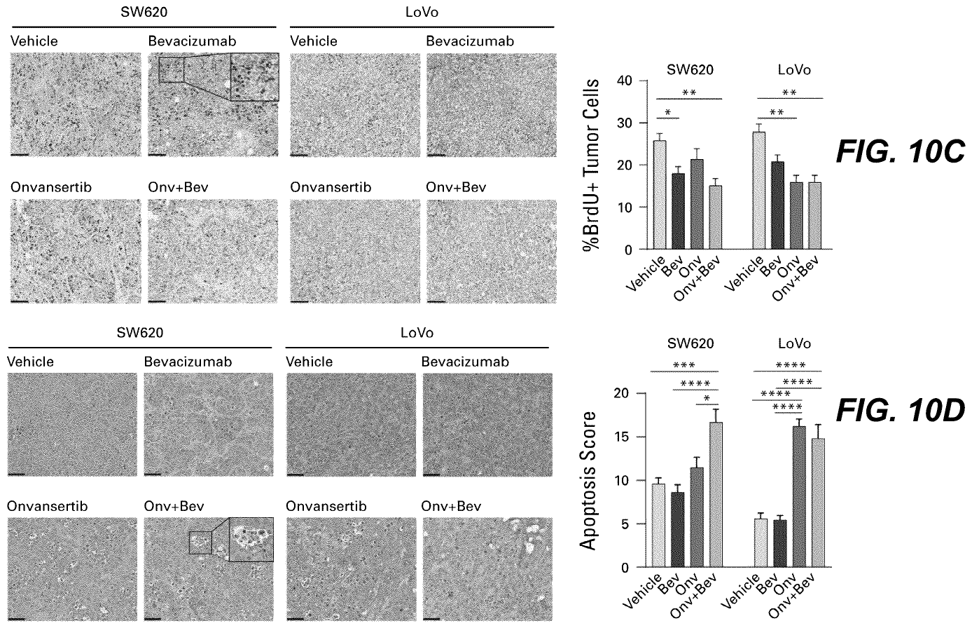

The antitumor activity of onvansertib and bevacizumab was evaluated in 3 KRAS mutant CRC xenograft models. The combination demonstrated robust antitumor activity across all 3 xenografts and superior inhibition of tumor growth compared to individual treatments (FIG. 7A). Tumor examination revealed reduced vascularization with onvansertib or bevacizumab single agents, and even greater reduction with the combination therapy (FIG. 7B, and FIG. 10A-FIG. 10D). These findings were corroborated through quantification of blood vessels using the endothelial marker CD31 (FIG. 7C). Furthermore, significant reduction in tumor cell proliferation and increase in apoptosis were observed with the combination (FIG. 10A-FIG. 10D).

### PLK1Regulates the Hypoxia Pathway in KRAS-Mutant CRC Cells

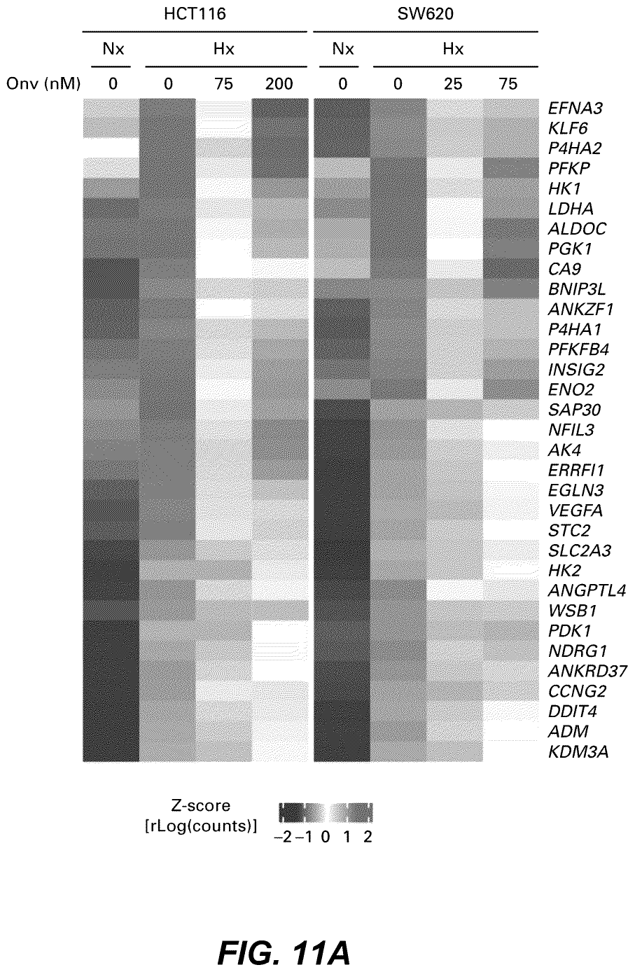

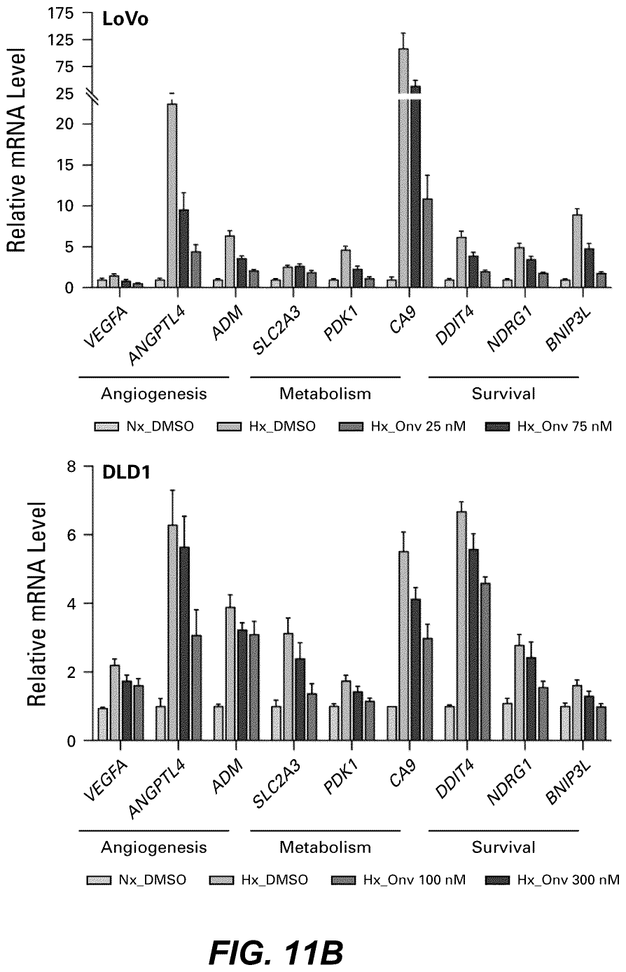

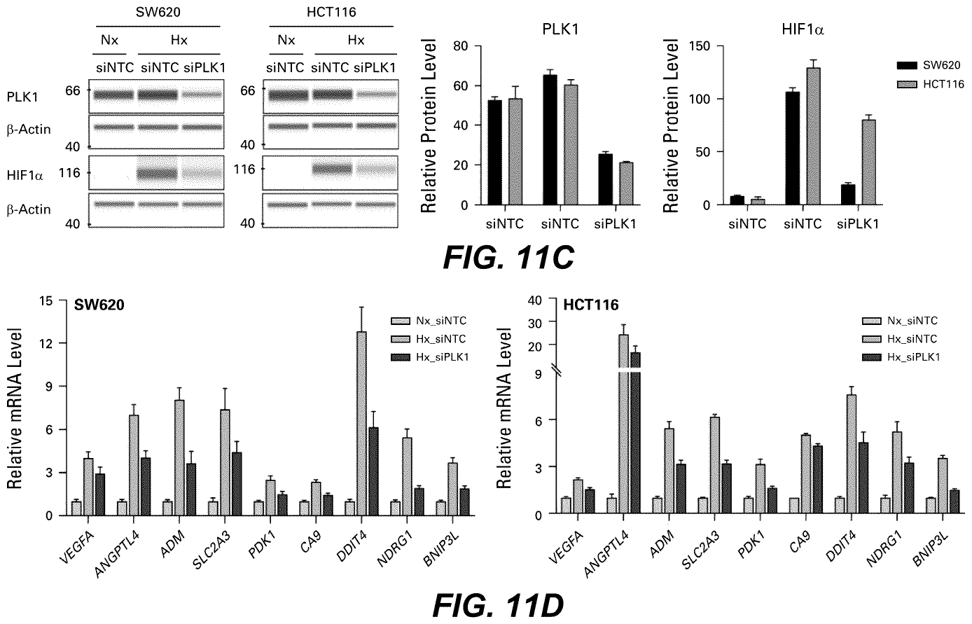

PLK1's role in angiogenesis was further explored. In tumors, hypoxia results in the stabilization of the hypoxia-inducible factor 1α (HIF1α), which triggers the expression of genes promoting angiogenesis, metabolic changes, and cell survival. Notably, onvansertib treatment led to a dose-dependent reduction in HIF1α protein expression in four KRAS-mutant CRC cell lines subjected to hypoxia (FIG. 7D). These data corroborated recent findings showing that PLK1 contributed to HIF1α stability in hypoxia. Transcriptomic profiling of HCT116 and SW620 cells exposed to hypoxia revealed that onvansertib significantly downregulated the hypoxia pathway and the closely associated metabolic pathway glycolysis in both cell lines (FIG. 7E). In the four cell lines, onvansertib inhibited the hypoxia pathway by preventing the induction of genes involved in angiogenesis, metabolic reprogramming, and cell survival (FIG. 11A-FIG. 11D). Similarly, depletion of PLK1 by siRNAs in HCT116 and SW620 cells subjected to hypoxia led to a marked decrease in both HIF1α protein and the mRNA expression of hypoxiarelated genes (FIG. 11A-FIG. 11D).

### DISCUSSION

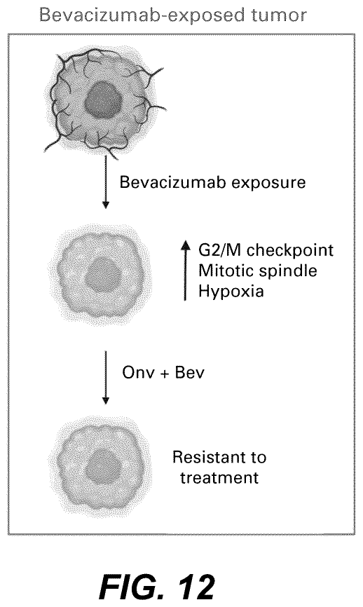

This is the first study to evaluate the combination of a PLK1 inhibitor with standard-of-care systemic therapy in second-line treatment of KRAS-mutant mCRC patients. The regimen of onvansertib+FOLFIRI and bevacizumab resulted in a confirmed ORR of 26.4%. Although this study fell short on meeting its primary endpoint of a 30% ORR, a bevacizumab-naïve subpopulation that achieved an ORR of 76.9% was unexpectedly identified. Based on these exceptional outcomes, the study was discontinued prematurely to focus on the clinical development of onvansertib for bevacizumab-naïve patients. This decision was supported by preclinical studies demonstrating onvansertib's impact on the hypoxia pathway and its combined effect with bevacizumab. Translational studies indicated that bevacizumab-treated tumors developed resistance mechanisms, likely impeding the synergy between onvansertib and bevacizumab (graphical summary provided in FIG. 1 and FIG. 12).

Combining chemotherapy and bevacizumab, the most commonly used second-line therapy in mCRC, has limited efficacy, with an ORR ranging between 5% and 23% and a median PFS of 6 to 7 months. Additionally, the presence of a KRAS mutation is associated with poorer outcomes. In comparison, this study showed encouraging efficacy with an ORR of 26.4% and median PFS of 8.4 months. Notably, all responses were confirmed and durable, with a median DOR of 11.7 months. The ORR of bevacizumab-naïve patients was 76.9% and their median PFS was 14.9 months, substantially exceeding any historical outcomes. In contrast, bevacizumab-exposed patients had an ORR of 10.0% and median PFS of 6.6 months, aligning with the expected outcomes for standard-of-care. Second-line regimens such as FOLFOX+bevacizumab or FOLFIRI+aflibercept evaluated in bevacizumab-naïve mCRC patients resulted in an ORR of approximately 20% and a median PFS of around 7 months, highlighting that the exceptional efficacy observed in this study in bevacizumab-naïve patients was unique to onvansertib.

Hypoxia activates adaptive cellular pathways through stabilization of the HIF transcription factors, promoting tumor survival, angiogenesis and metastasis and thereby contributing to treatment resistance. The preclinical studies in this example demonstrated that onvansertib inhibited the hypoxia pathway by downregulating HIF1α protein, likely contributing to reduced tumor vascularization and robust antitumor activity of onvansertib and bevacizumab. Although dual inhibition of angiogenesis has the potential to enhance antitumor effects and delay resistance, combining bevacizumab with other antiangiogenic agents like sunitinib has proven challenging due to high toxicities. In contrast, the combination of onvansertib with FOLFIRI and bevacizumab was well tolerated, with no increase in vascular toxicities beyond those expected from bevacizumab alone (e.g., hypertension, bleeding, clotting), supporting the hypothesis that onvansertib's antiangiogenic activity is mediated through tumor-cell-specific pathways, sparing normal vasculature. Onvansertib also downregulated glycolysis-related genes disrupting another critical survival pathway in cancer cells. Glycolysis is essential for supporting energy needs, biosynthetic processes, and adaption to the hypoxic microenvironment in tumor cells, especially those with oncogenic KRAS mutations. By targeting the hypoxia pathway, onvansertib can inhibit tumor growth through dual mechanisms, it can collaborate with bevacizumab to block angiogenesis and inhibit crucial tumor survival pathways such as glycolysis, offering a promising two-hit therapeutic strategy against KRAS-mutant CRC. Further investigations are warranted to determine whether this strategy is unique to mCRC tumors harboring KRAS mutations.

Resistance to targeted therapies can involve the activation of compensatory signaling pathways. For instance, adaptive resistance to KRAS^{G12C} inhibitors can occur via EGFR-mediated reactivation of the MAPK pathway and combining EGFR and KRAS^{G12C} inhibitors has shown to enhance antitumor activity. The clinical data in this example suggest that first-line treatment with bevacizumab resulted in resistance to onvansertib. Real-world data analysis indicated that bevacizumab-exposed tumors from KRAS-mutant mCRC patients exhibited an upregulation of pathways related to PLK1 functions in mitosis, potentially contributing to onvansertib resistance. Further validations using independent datasets and experimental testing are warranted to corroborate this hypothesis.

There are several limitations to this clinical study to consider. Firstly, this was a single-arm study with no control group. Secondly, the study was terminated before reaching its estimated sample size, resulting in reduced statistical power. Finally, the increased clinical benefit observed in bevacizumab-naïve patients was based on a post hoc analysis of a small subset of patients, underscoring the need for further validation through larger and randomized controlled trials.

In conclusion, this study demonstrated the efficacy and tolerability of onvansertib+FOLFIRI and bevacizumab in second-line treatment of KRAS-mutant mCRC patients. Patients with no prior exposure to bevacizumab showed marked increased efficacy with an ORR of 76.9% and a median PFS of 14.9 months, underscoring the unique sensitivity of this population to onvansertib. This exceptional response, alongside our translational studies showing onvansertib's effect on the hypoxia pathway and angiogenesis, supported shifting onvansertib development to the front-line setting, where all patients are bevacizumab naïve. CRDF-004 (ClinicalTrails.gov identifier: NCT06106308) is an openlabel, multicenter, randomized phase II study, currently enrolling to evaluate the efficacy and tolerability of first-line onvansertib+chemotherapy+bevacizumab versus chemotherapy+bevacizumab in patients with RAS-mutant mCRC.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treating a metastatic colorectal cancer, comprising:
administering to a patient with a metastatic colorectal cancer (mCRC) an effective amount of onvansertib and an effective amount of bevacizumab, wherein the patient has not received any treatment comprising bevacizumab within at least three months prior to the administration of onvansertib and bevacizumab.

2. The method of claim 1, wherein the patient has not received any treatment comprising bevacizumab within about three months or about four months prior to the administration of onvansertib and bevacizumab.

3. The method of claim 1, wherein the patient has not received any treatment comprising bevacizumab within about three months or about twelve months prior to the administration of onvansertib and bevacizumab.

4. The method of claim 1, wherein the patient has received at least one treatment comprising bevacizumab more than three months prior to the administration of onvansertib and bevacizumab.

5. The method of any one of claim 1, wherein the patient has received at least one treatment not comprising bevacizumab.

6. The method of claim 5, wherein the at least one treatment not comprising bevacizumab occurs within about three months prior to the administration of onvansertib and bevacizumab.

7. The method of any one of claim 1, further comprising, at least three months after the administration of onvansertib and bevacizumab has ended, administrating at least one additional treatment comprising bevacizumab to the patient.

8. The method of claim 7, wherein the at least one additional treatment comprising bevacizumab and onvansertib.

9. The method of claim 7, wherein any two additional treatments comprising bevacizumab are separated by a time period of at least three months.

10. The method of any one of claim 1, wherein the patient has not received any treatment comprising bevacizumab and a chemotherapy containing fluorouracil within at least three months prior to the administration of onvansertib and bevacizumab.

11. The method of any one of claim 1, wherein the metastatic colorectal cancer is a mutated metastatic colorectal cancer having one or more mutations in the RAS gene.

12. The method of claim 11, wherein the metastatic colorectal cancer is KRAS-mutated metastatic colorectal cancer, NRAS-mutated metastatic colorectal cancer, HRAS-mutated metastatic colorectal cancer, or a combination thereof.

13. The method of any one of claim 1, wherein administration of onvansertib and bevacizumab is performed in combination with a chemotherapy containing fluorouracil.

14. The method of any one of claim 1, wherein the administration comprises at least two treatment cycles of the administration of onvansertib and bevacizumab, and optionally wherein two of the treatment cycles are in consecutive.

15. The method of claim 14, wherein onvansertib is administered on at least four days in a treatment cycle and bevacizumab is administered once every week or two weeks during a treatment cycle.

16. The method of any one of claim 14, wherein a treatment cycle is about 21 to 28 days.

17. The method of any one of claim 1, wherein onvansertib and bevacizumab are administered to the patient simultaneously, separately, or sequentially.

18. The method of any one of claim 1, wherein the administration of onvansertib is oral administration, and the administration of bevacizumab is intravenous administration.

19. The method of any one of claim 1, wherein onvansertib is administered at 12 mg/m²-90 mg/m², optionally at 15 mg/m²-30 mg/m².

20. The method of any one of claim 1, wherein onvansertib is administered at 5-50 mg; optionally at 10-30 mg; and further optionally at 10 mg, 15 mg, 20 mg, 25 mg, or 30 mg.

21. The method of claim 1, wherein bevacizumab is administered at 1 mg/kg-20 mg/kg.

22. The method of claim 1, wherein onvansertib and bevacizumab are administered to the patient in combination with FOLFIRI or FOLFOX.

23. The method of claim 1, further comprising identifying the patient as having the metastatic colorectal cancer and as not having received any treatment comprising bevacizumab within at least three months prior to the administration of onvansertib and bevacizumab.

24. The method of claim 1, further comprising excluding patients who have received a prior treatment comprising bevacizumab within at least three months from receiving the onvansertib and bevacizumab treatment.

25. The method of claim 24, further comprising identifying patients who have received a prior treatment comprising bevacizumab within at least three months.
